(19) United States
(12) Patent Application Publication
(10) Pub. No.: US 2005/0209295 A1
(43) Pub. Date: Sep. 22, 2005

(54) Methimazole derivatives and tautomeric cyclic thiones to inhibit cell adhesion

(76) Inventors: Leonard D. Kohn, Athens, OH (US); Douglas Goetz, Athens, OH (US); Norikazu Hari, Yaminashi (JP); Chris Lewis, Athens, OH (US); Cesidio Giuliani, Roccamorice (IT); Giorgio Napolitano, Pescara (IT); Nilesh Dagia, Athens, OH (US)

Correspondence Address: FROST BROWN TODD, LLC, 2200 PNC CENTER, 201 E. FIFTH STREET, CINCINNATI, OH, 45202, US

(21) Appl. No.: 10/801,986

(22) Filed: Mar. 16, 2004

(51) Int. Cl. A61K 31/4166

(52) U.S. Cl. 514/389

(57) ABSTRACT

The present invention relates to novel compounds and methods of use for inhibition and prevention of cell adhesion and cell adhesion-mediated pathologies. This invention also relates to pharmaceutical formulations comprising these compounds and methods of using them for inhibition and prevention of cell adhesion and cell adhesion-mediated pathologies. The compounds and pharmaceutical compositions of this invention can be used as therapeutic or prophylactic agents. In particular, methimazole derivatives and tautomeric cyclic thiones have the ability to inhibit the adhesion and the migration of leukocytes. In addition to being active anti-inflammatories, the methimazole derivatives and tautomeric cyclic thiones and their physiologically tolerable salts, derivatives and prodrugs are generally suitable for the treatment (i.e., for the therapy and prophylaxis) of diseases that are based on the interaction between VCAM-1 and its ligands or can be influenced by an inhibition of this interaction. In particular, the methimazole derivatives and tautomeric cyclic thiones are suitable for the treatment of diseases that are caused at least partly by an undesired extent of leukocyte adhesion and/or leukocyte migration or are connected therewith, and for whose prevention, alleviation or cure the adhesion and/or migration of leukocytes should be decreased.

This invention was made with government support under Grant Nos. BES 9733542 (0096303) awarded by the National Science Foundation and GM57640 (DJG) awarded by the National Institutes of Health. The government may have certain rights in the invention.

### TECHNICAL FIELD

The present invention relates to novel compounds and methods of use for inhibition and prevention of cell adhesion and cell adhesion-mediated pathologies. This invention also relates to pharmaceutical formulations comprising these compounds and methods of using them for inhibition and prevention of cell adhesion and cell adhesion-mediated pathologies.

### BACKGROUND OF THE INVENTION

Cell adhesion is a process by which cells associate with each other, migrate towards a specific target or localize within the extra-cellular matrix. As such, cell adhesion constitutes one of the fundamental mechanisms underlying numerous biological phenomena. For example, cell adhesion is responsible for the adhesion of hematopoietic cells to endothelial cells and the subsequent migration of those hematopoietic cells out of blood vessels and to the site of injury. Cell adhesion plays a role in pathological inflammation and immune reactions in mammals.

Adhesion mediated by VCAM-1 and other endothelial cell surface receptors is associated with a number of inflammatory responses. At the site of an injury or other inflammatory stimulus, activated vascular endothelial cells express molecules that are adhesive for leukocytes. The mechanics of leukocyte adhesion to endothelial cells involves, in part, the recognition and binding of cell surface receptors on leukocytes to the corresponding cell surface molecules on endothelial cells. Once bound, the leukocytes migrate across the blood vessel wall to enter the injured site and release chemical mediators to combat infection.

Inflammatory brain disorders, such as experimental autoimmune encephalomyelitis (EAE), multiple sclerosis (MS) and meningitis, are examples of central nervous system disorders in which the endothelium/leukocyte adhesion mechanism results in destruction to otherwise healthy brain tissue. Large numbers of leukocytes migrate across the blood brain barrier (BBB) in subjects with these inflammatory diseases. The leukocytes release toxic mediators that cause extensive tissue damage resulting in impaired nerve conduction and paralysis.

In other organ systems, tissue damage also occurs via an adhesion mechanism resulting in migration or activation of leukocytes. For example, it has been shown that the initial insult following myocardial ischemia to heart tissue can be further complicated by leukocyte entry to the injured tissue causing still further insult. Other pathologies mediated by an adhesion mechanism include, by way of example, asthma, Alzheimer's disease, atherosclerosis, AIDS dementia, diabetes, inflammatory bowel disease, multiple sclerosis, rheumatoid arthritis, tissue transplantation and tumor metastasis.

The adhesion of leukocytes to the endothelium in the fluid dynamic environment of the circulation plays a central role in pathological inflammation (e.g. atherosclerosis (1), and inflammatory bowel disease (2)). Endothelial cell adhesion molecules (ECAMs³) known to participate in leukocyte recruitment, (e.g. VCAM-1, E-selectin and ICAM-1), have been shown to be up-regulated in such settings and to contribute to disease progression and/or tissue damage by virtue of their role in leukocyte adhesion (3). For example, VCAM-1 is present in a localized fashion on aortic endothelium that overlies early foam cell lesions (1) and is increased on endothelium in models of colitis (4). A promising therapeutic approach for treating pathological inflammation is, therefore, to reduce aberrant leukocyte adhesion to the endothelium via suppression of ECAM expression (5).

ECAM expression is influenced by the cytokine milieu in which the endothelial cells reside. Indeed, treating cultured endothelial cells with the pro-inflammatory cytokine TNF-α for 4 hrs. elicits expression of E-selectin, VCAM-1 and ICAM-1 (6). The cytokine dependent ECAM induction is regulated at the gene level by the activity of transcription factors such as nuclear factor-κB (NF-κB), activator protein-1 (AP-1), specificity protein-1 (SP-1), interferon regulatory factor-1 (IRF-1) and GATA. For example, the E-selectin promoter has binding sites for NF-κB (7), the VCAM-1 promoter has binding sites for NF-κB, AP-1, SP-1, IRF-1 and GATA (8-11) and the ICAM-1 promoter has functional binding sites for NF-κB and AP-1 (12, 13). Some of these transcription factors (e.g. NF-κB) are present in unstimulated endothelial cells in an inactive form (14). Cytokine treatment of endothelial cells stimulates the activity of these transcription factors (14) and also induces the expression of other transcription factors (e.g. IRF-1) (10). The active/induced transcription factors ligate to their respective binding sites leading to gene transcription.

Several current or potential therapeutics for pathological inflammation work, at least in part, by modulating the activity of transcription factors (15-19). Indeed, compounds that block cytokine induced ECAM expression at the transcription level have been shown to inhibit leukocyte adhesion to the endothelium (16-18, 20) and to reduce inflammation in animal models (15, 17). Methimazole is widely used clinically for the treatment of autoimmune Graves' disease or primary hyperthyroidism (21) and has been shown to be effective in treating several other forms of autoimmune disease, both psoriasis in humans (22) and systemic lupus, autoimmune blepharitis, autoimmune uveitis, thyroiditis, and diabetes in murine experimental models (23-26). Evidence has accumulated that methimazole acts as a transcriptional inhibitor of abnormally increased MHC Class I and Class II gene expression (26-29) and mimics the effect of a Class I knockout in preventing autoimmune disease (30). Several observations suggest that methimazole may also affect ECAM expression and thus could be a potential anti-inflammatory compound. Specifically, it has been reported that (a) Graves' disease patients treated with methimazole have reduced levels of circulating soluble E-selectin and soluble VCAM-1 (31) and (b) methimazole decreases colonic mucosal damage in a rat model of experimental colitis (32). An effort to identify derivative compounds with greater efficacy as an anti-immune agent or immunosuppressive than methimazole, led to the observation that phenyl methimazole (compound 10, C-10), a tautomeric cyclic thione, was 50 to 100-fold more potent at suppressing abnormally increased MHC gene expression and was a far more effective agent in experimental models of lupus and diabetes (26, 28). These observations motivated us to probe the hypothesis that a derivative of methimazole, phenyl methimazole, or other tautomeric cyclic thiones can reduce pro-inflammatory cytokine (e.g. TNF-α)-induced ECAM expression and consequent leukocyte adhesion to endothelial cells.

There remains a need for inhibitors of VCAM-1-dependent cell adhesion. Such compounds would provide useful agents for treatment, prevention or suppression of various pathologies involving VCAM-1 mediated cell adhesion.

It has now been found that a specific class of methimazole derivatives and tautomeric cyclic thiones are effective as anti-inflammatories, in the case of inflammatory symptoms of very different cause, to prevent, reduce or suppress the undesired or harmful sequence of the inflammation. They are used, for example, for the treatment of arthritis, rheumatoid arthritis, polyarthritis, inflammatory bowel disease (ulcerative colitis, Crohn's disease), systemic lupus erythematosus, inflammatory diseases of the central nervous system (e.g., multiple sclerosis), or asthma or allergies (e.g., allergies of the delayed type (type IV allergy)). Furthermore, compounds of the present invention are suitable for cardioprotection, for stroke protection and for the secondary prophylaxis of stroke and for the treatment of cardiovascular diseases, atherosclerosis, myocardial infarct, myocardial reinfarct, acute coronary syndrome, stroke, restenoses, diabetes, damage to organ transplants, immune diseases, autoimmune diseases, tumor growth or tumor metastasis in various malignancies, malaria and other diseases where a blocking of abnormally increased expression of VCAM-1 and/or an influencing of the leukocyte activity appears appropriate for prevention, alleviation or cure. A preferred use is the prevention of inflammatory bowel disease and macro- or microvascular complications of Types I or II diabetes, e.g., myocardial infarct or of myocardial reinfarct or nephropathy.

### SUMMARY OF THE INVENTION

The present invention relates to novel compounds and methods of use for inhibition and prevention of cell adhesion and cell adhesion-mediated pathologies. This invention also relates to pharmaceutical formulations comprising these compounds and methods of using them for inhibition and prevention of cell adhesion and cell adhesion-mediated pathologies. The compounds and pharmaceutical compositions of this invention can be used as therapeutic or prophylactic agents. In particular, methimazole derivatives and tautomeric cyclic thiones have the ability to inhibit the adhesion and the migration of leukocytes (e.g., the adhesion of monocytes to endothelial cells), which is mediated by VCAM-1 adhesion mechanism. In addition to being active anti-inflammatories, the methimazole derivatives and tautomeric cyclic thiones and their physiologically tolerable salts and derivatives are generally suitable for the treatment (i.e., for the therapy and prophylaxis) of diseases that are based on the interaction between VCAM-1 and VCAM-1 ligands (e.g. VLA-4 or α4β7) or can be influenced by an inhibition of this interaction. In particular, the methimazole derivatives and tautomeric cyclic thiones are suitable for the treatment of diseases that are caused at least partly by an undesired extent of leukocyte adhesion and/or leukocyte migration or are connected therewith, and for whose prevention, alleviation or cure the adhesion and/or migration of leukocytes should be decreased.

The present invention also relates to the methimazole derivatives and tautomeric cyclic thiones and/or their physiologically acceptable salts and/or derivatives for the inhibition of the adhesion and/or migration of leukocytes or for the inhibition of abnormally increased VCAM-1 expression, e.g. that induced by cytokines such as TNF-α. In addition, the present invention relates to the use of the methimazole derivatives and tautomeric cyclic thiones and/or their physiologically acceptable salts and/or derivatives for the preparation of pharmaceuticals thereof, i.e., of pharmaceuticals for the treatment of diseases, wherein the leukocyte adhesion and/or leukocyte migration shows an undesired extent, or for the treatment of diseases, wherein VCAM-1-dependent adhesion processes play a role, and to the use of the methimazole derivatives and tautomeric cyclic thiones and/or their physiologically acceptable salts and/or derivatives in the treatment of diseases of this type.

In one embodiment, the present invention provides for methods for reducing aberrant leukocyte-endothelial adhesion during pathological inflammation by inhibiting endothelial cell adhesion molecule (ECAM) expression at the transcriptional level. Specifically, the present invention provides for methods of using methimazole derivatives to modulate TNF-α-induced ECAM (e.g., E-selectin, ICAM-1 and VCAM-1) expression and consequent monocytic cell (U-937) adhesion to human aortic endothelial cells (HAEC).

According to one embodiment of this invention, these novel compounds, compositions and methods are advantageously used to treat inflammatory and immune diseases. The present invention also provides methods for preparing the compounds of this invention and intermediates useful in those methods.

In one embodiment, the present invention provides for the use of methimazole (1-methyl-2-mercaptoimidazole) and its derivatives. In another embodiment, the present invention provides for the use of a prodrug form of methimazole, known as carbimazole (neomercazole) and its derivatives.

In another embodiment, the present invention provides for the use of a composition containing one or more of the compounds selected from the group consisting of methimazole, metronidazole, 2-mercaptoimidazole, 2-mercaptobenzimidazole, 2-mercapto-5-nitrobenzimidazole, 2-mercapto-5-methylbenzimidazole, s-methylmethimazole, n-methylmethimazole, 5-methylmethimazole, 5-phenylmethimazole, and 1-methyl-2-thiomethyl-5(4)nitroimidazole. Preferably, 5-phenylmethimazole is used.

In another embodiment, the present invention provides for the use of phenyl methimazole (compound 10; C-10) and its derivatives.

It is one objective of the present invention to provide methods of using phenyl methimazole to modulate or reduce TNF-α-induced monocytic cell adhesion to HAEC by inhibiting VCAM-1 gene expression in an IRF-1 dependent manner.

Compounds of this invention may be synthesized using any conventional technique. Preferably, these compounds are chemically synthesized from readily available starting materials.

The compounds of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

As used throughout this application, the term “patient” refers to mammals, including humans. And the term “cell” refers to mammalian cells, including human cells.

Once synthesized, the activities and VCAM-1 specificities of the compounds according to this invention may be determined using in vitro and in vivo assays.

For example, the cell adhesion inhibitory activity of these compounds may be measured by determining the concentration of inhibitor required to block the binding of VCAM-1-expressing cells to VCAM-1 ligand (e.g., VLA-4) expressing cells (e.g., monocytes, lymphocytes). In this assay, microtiters wells are coated with cells (e.g., endothelial cells), which can express VCAM-1. Once the wells are coated, varying concentrations of the test compound are then added together with a cytokine (e.g., TNF-α), which can induce the expression of VCAM-1. Alternatively, the test compound may be added first and allowed to incubate with the coated wells containing endothelial cells prior to the addition of the cytokine. The cells are allowed to incubate in the wells for at least 2 hrs. Following incubation, appropriately labeled VCAM-1 ligand-expressing cells (e.g., monocytes, lymphocytes) are added to the wells and incubated for at least 30 minutes. After the incubation period, the wells are washed. Inhibition of binding is measured by quantitating the fluorescence or radioactivity bound to the VCAM-1 expressing cells in the plate for each of the various concentrations of test compound, as well as for controls containing no test compound.

VCAM-1-expressing cells that may be utilized in this assay include nonimmune target tissue cells, endothelial cells, and epithelial cells. The VCAM-1 ligand expressing cells (e.g., monocytes, lymphocytes) used in this assay may be fluorescently or radioactively labeled.

A direct binding assay may also be employed to quantitate the inhibitory activity of the compounds of this invention.

Once VCAM-1-specific inhibitors are identified, they may be further characterized in in vivo assays. One such assay tests the effects of inhibitors on VCAM-1 expression and leukocyte adhesion in well-established in vivo models of pathological inflammation (e.g., inflamed mesenteric endothelium in murine model of chronic inflammation (i.e., colitis); isolated carotid arteries of apolipoprotein E-deficient (apoE −/−) mice with developing atherosclerotic lesions).

The compounds of the present invention may be used in the form of pharmaceutically acceptable salts derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The compounds of the present invention may be formulated into pharmaceutical compositions that may be administered orally, parenterally, by inhalation spray, topically, rectally, nasally, buccally, vaginally or via an implanted reservoir. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques.

The pharmaceutical compositions of this invention comprise any of the compounds of the present invention, or pharmaceutically acceptable salts thereof, together with any pharmaceutically acceptable carrier. The term “carrier” as used herein includes acceptable adjuvants and vehicles. Pharmaceutically acceptable carriers that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. They may comprise liposomes or drug carriers made lipids or polymeric particles, including biodegradable polymers, or targeted delivery applications, e.g., coupling to antibodies. They include solubilization with dimethylsulfoxide before dilution to final useful concentrations.

According to this invention, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol or dimethyl sulfoxide. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as do natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at the rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient that may be combined with the carrier materials to produce a single dosage form will vary depending upon the host treated, and the particular mode of administration. It should be understood, however, that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredient may also depend upon the therapeutic or prophylactic agent, if any, with which the ingredient is co-administered.

The dosage and dose rate of the compounds of this invention effective to prevent, suppress or inhibit cell adhesion will depend on a variety of factors, such as the nature of the inhibitor, the size of the patient, the goal of the treatment, the nature of the pathology to be treated, the specific pharmaceutical composition used, and the judgment of the treating physician. Dosage levels of between about 0.001 and about 100 mg/kg body weight per day, preferably between about 0.1 and about 10 mg/kg body weight per day of the active ingredient compound are useful.

According to another embodiment compositions containing a compound of this invention may also comprise an additional agent selected from the group consisting of corticosteroids, bronchodilators, antiasthmatics (mast cell stabilizers), anti-inflammatories, antirheumatics, immunosuppressants, antimetabolites, immunonodulators, antipsoriatics, antibiotics, and antidiabetics. Also included within this group are compounds such as theophylline, sulfasalazine and aminosalicylates (antiinflammatories); cyclosporin, FK-506, and rapamycin (immunosuppressants); cyclophosphamide and methotrexate (antimetabolites); and interferons (immunomodulators).

According to other embodiments, the invention provides methods for preventing, inhibiting or suppressing cell adhesion-associated inflammation and cell adhesion-associated immune or autoimmune responses. VCAM-1-associated cell adhesion plays a central role in a variety of inflammatory, immune and autoimmune diseases. Thus, inhibition of cell adhesion by the compounds of this invention may be utilized in methods of treating or preventing inflammatory, immune and autoimmune diseases. Preferably the diseases to be treated with the methods of this invention are selected from asthma, arthritis, psoriasis, transplantation rejection, multiple sclerosis, diabetes, inflammatory bowel disease, and inflammatory/immune diseases related to activation of the innate immune system such as endotoxic shock.

These methods may employ the compounds of this invention in a monotherapy or in combination with an anti-inflammatory or immunosuppressive agent. Such combination therapies include administration of the agents in a single dosage form or in multiple dosage forms administered at the same time or at different times.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

Throughout this document, all temperatures are given in degrees Celsius, and all percentages are weight percentages unless otherwise stated. All publications mentioned herein are incorporated herein by reference for the purpose of describing and disclosing the compositions and methodologies, which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

### BRIEF DESCRIPTION OF THE DRAWINGS

This invention, as defined in the claims, can be better understood with reference to the following drawings:

FIG. 1. A methimazole derivative (C-10) significantly inhibits short term (2-4 hr.) TNF-α induced expression of VCAM-1 but has little to no effect on TNF-α induced E-selectin and ICAM-1 expression. The protein (A) and mRNA (B) levels of E-selectin, ICAM-1, VCAM-1 on unactivated and 4 (ELISA) or 2 (mRNA) hr. TNF-α activated HAEC, in the absence or presence of C-10, are determined by ELISA and Northern blot analysis respectively. (A) The level of absorbance indicated by optical density (O.D. at 450 nm) correlates with the level of a given ECAM protein (e.g. E-selectin) on the HAEC. All values are mean+/−std. deviation of triplicate wells. Results presented are representative of a typical experiment done at least 3 times. A mAb to LFA-1 (TS1/22) served as a negative control. (B) RNA isolated from HAEC was subjected to Northern blot analyses using appropriate probes for E-selectin, ICAM-1 and VCAM-1. G3PDH probe was used as the loading control. Results presented are typical of 3 separate experiments. (Legend: TNF-α activation indicates activation of HAEC with TNF-α (+) or no activation (−) prior to the assay. Treatment indicates treatment of HAEC with C-10, DMSO or no treatment (−) during activation with TNF-α. * indicates p<0.001).

FIG. 2. C-10 significantly inhibits 24 hr. TNF-α induced expression of VCAM-1 and E-selectin but has no effect on ICAM-1 expression. The protein (A) and mRNA (B) levels of E-selectin, ICAM-1, VCAM-1 on unactivated and 24 hr. TNF-α activated HAEC, in the absence or presence of C-10, are determined by ELISA and Northern blot analysis respectively. (A) The level of absorbance indicated by optical density (O.D. at 450 nm) correlates with the level of a given ECAM protein (e.g. E-selectin) on the HAEC. All values are mean+/−std. deviation of triplicate wells. Results presented are representative of a typical experiment done at least 3 times. A mAb to LFA-1 (TS1/22) served as a negative control and gave results (data not shown) similar to that shown in FIG. 1A. (B) RNA isolated from HAEC was subjected to Northern blot analyses using appropriate probes for E-selectin, ICAM-1 and VCAM-1. G3PDH probe was used as a loading control. Results presented are typical of 3 separate experiments. (Legend: TNF-α activation indicates activation of HAEC with TNF-α (+) or no activation (−) prior to the assay. Treatment indicates treatment of HAEC with C-10, DMSO or no treatment (−) during activation with TNF-α. * indicates p<0.001)

FIG. 3. C-10 has a modest effect on U937 adhesion to 4 hr. TNF-α activated HAEC and a dramatic effect on U937 adhesion to 24 hr. TNF-α activated HAEC. HAEC are treated for 4 hrs. (A) or 24 hrs. (B) with TNF-α in the absence or presence of C-10. In certain instances, HAEC are pre-treated with mAb prior to use in adhesion assays. Subsequently, U937 cells are perfused over the HAEC and the number of U937 cells adherent to the HAEC at the end of 2.5 minutes of flow determined. (Legend: TNF-α activation indicates activation of HAEC with TNF-α (+) or no activation (−) for 4 hrs. (A) or 24 hrs. (B) prior to the adhesion assay. Treatment indicates treatment of HAEC with 0.5 mM (A) or 0.1 mM (B) C-10, DMSO or no treatment (−) during activation with TNF-α mAb indicates pre-treatment of HAEC with a mAb to E-selectin, HEL3/2 (E), a mAb to VCAM-1,51-10C9 (V), a combination of mAbs to VCAM-1, 51-10C9, and E-selectin, HEL3/2 (V+E) or no pre-treatment (−) after the other treatments but prior to the adhesion assay. Shear Stress=1.8 dynes/cm²; n>3; error bars represent SEM; # indicates p<0.05)

FIG. 4. C-10 inhibits TNF-α induced increase in VCAM-1 promoter activity in HAEC. (Left) The locations of the binding sites for various transcription factors known to play a role in TNF-α induced human VCAM-1 expression are used as a template to create −1641/+12, −288/+12, −228/+12 and −85/+12 bp constructs. (Right) HAEC are transfected for 24 hrs. with 400 ng of the constructs indicated on the left or pGL3 basic luciferase reporter vector. All HAEC are also transfected with phRL-TK (Int−) vector that contains Renilla luciferase (Rluc) as an “internal” transfection control. HAEC are treated for 6 hrs. with 10 ng/ml TNF-α in the absence or presence of 0.3 mM C-10. All treatment conditions contained 0.3% DMSO. Assays are conducted with the Dual-Luciferase Reporter Assay System. The luciferase activity indicated by relative light units (R.L.U) correlates with the level of promoter activity. All values are mean+/−std. deviation of triplicate wells. Results presented are typical of 3 separate experiments. (Legend: Bar 1 indicates untreated HAEC, bar 2 indicates 0.3 mM C-10 treated HAEC, bar 3 indicates 10 ng/ml TNF-α treated HAEC and bar 4 indicates HAEC treated with 10 ng/ml TNF-α in the presence of 0.3 mM C-10.)

FIG. 5. C-10 inhibits 2 hr. TNF-α induced IRF-1 binding activity to VCAM-1 promoter. (A) Probe sequences used in EMSA. Overhead line indicates the IRF-1 binding site. Lower case letters indicate the mutated bases. The sense strand sequence of the consensus NF-κB probe (not shown) is: 5′ AGTTGAGGGGACTTTCCCAGGC 3′. (B) EMSA or (C) supershift EMSA are performed with ³²P-labeled IRF-1 probe and 3 μg of nuclear extracts prepared from HAEC treated with or without 10 ng/mL TNF-α in the absence or presence of C-10. Results presented are typical of 2 separate experiments. (Legend: TNF-α activation indicates activation of HAEC with TNF-α (+) or no activation (−) prior to the assay. Treatment indicates treatment of HAEC with 0.5 mM C-10, 1 mM C-10, or DMSO (DMSO) or no treatment (−) during activation with TNF-α. Competitor indicates absence (−) or presence of 100 fold molar excess of unlabeled VCAM-1 IRF-1 probe (VCAM-1 IRF wild) or VCAM-1 IRF-1 mutant probe (VCAM IRF Mutant) or consensus IRF-1 probe (Cons. IRF) or consensus NF-κB probe (cons. NF-κB) or 2 μg antibody to IRF-1 (IRF-1 Ab)).

FIG. 6. C-10 reduces TNF-α induced IRF-1 expression. The mRNA (A) and protein (B) levels of IRF-1 on unactivated and 2 hr. TNF-α activated HAEC, in the absence or presence of C-10, are determined by Northern blot analysis and Western blot analysis respectively. (A) RNA isolated from HAEC was subjected to Northern blot analyses using IRF-1 probe. Ethidium bromide stained rRNA was the loading control. (B) Whole cell lysates of HAEC are subjected to Western blot analysis using IRF-1 antibody. Ponceau S staining of blots after transfer revealed equivalent loading of total protein (data not shown). (Legend: TNF-α indicates activation of HAEC with 10 ng/ml TNF-α (+) or no activation (−) prior to the assay. C-10 indicates treatment of HAEC with 0.5 mM or 1 mM C-10 or DMSO (−) during activation with TNF-α.)

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

### DETAILED DESCRIPTION OF THE INVENTION

Before the present device and methods for tissue augmentation is described, it is to be understood that this invention is not limited to the specific methodology, devices, formulations, and compositions described as such may, of course, vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “and”, and “the” include plural referents unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

The present invention also provides for the use of methimazole derivatives and tautomeric cyclic thione compounds that specifically inhibit the binding of VCAM-1. These compounds are useful for inhibition, prevention and suppression of VCAM1-mediated cell adhesion and pathologies associated with that adhesion, such as inflammation and immune reactions. The compounds of this invention may be used alone or in combination with other therapeutic or prophylactic agents to inhibit, prevent or suppress cell adhesion. This invention also provides pharmaceutical formulations containing these VCAM-1-mediated cell adhesion inhibitors and methods of using the compounds and compositions of the invention for inhibition of cell adhesion. As used herein, the following terms shall have the definitions given below.

The phrase “safe and effective amount” means a sufficient amount of pharmaceutically active compound to effect the inhibition and prevention of cell adhesion and cell adhesion-mediated pathologies. Within the scope of sound medical judgement, the required dosage of a pharmaceutically active agent or of the pharmaceutical composition containing that active agent will vary with the severity of the condition being treated, the duration of the treatment, the nature of adjunct treatment, the age and physical condition of the patient, the specific active compound employed, and like considerations discussed more fully hereinafter. In arriving at the “safe and effective amount” for a particular compound, these risks must be taken into consideration, as well as the fact that the compounds described herein provide pharmaceutical activity at lower dosage levels than conventional methimazole compounds.

“Pharmaceutically-acceptable” shall mean that the pharmaceutically active compound and other ingredients used in the pharmaceutical compositions and methods defined herein are suitable for use in contact with the tissues of humans and lower animals without undue toxicity, irritation, allergic response, and the like, commensurate with a reasonable benefit/risk ratio.

The term “administration” of the pharmaceutically active compounds and the pharmaceutical compositions defined herein includes systemic use, as by injection (especially parenterally), intravenous infusion, suppositories and oral administration thereof, as well as topical application of the compounds and compositions. Oral administration is particularly preferred in the present invention.

“Ameliorate” or “amelioration” means a lessening of the detrimental effect or severity of the cell adhesion disorder in the subject receiving therapy, the severity of the response being determined by means that are well known in the art.

The term adhesion disorder of cell adhesion means atypical cell adhesion leading to pathologies, including, without limitation, reperfusion injury following ischemia, atherosclerosis, inflammatory bowel disease (Crohns' disease and ulcerative colitis), thermal injury (burns), arthritis, asthma, organ transplantation (host vs. graft and graft vs. host), stroke, malaria, multiple sclerosis, diabetes, hemorrhagic shock, myocardial infarcts, pulmonary infarcts, cerebral infarcts, intestinal infarcts, renal infarcts, sepsis, thrombosis, delayed type hypersensitivity reaction, cancer, acute lung injury, autoimmune or nonautoimmune glomerulonephritis, tuberculosis, sarcoidosis, systemic lupus erythematosus, Sjogren's disease, polymyositis/dermatomyositis, hypertensive vascular disease, vasculitis, polyarteritis nodosa, giant cell arteritis, Wegner's granulomatosis, Kawasaki's disease (mucocutaneous lymph node syndrome), thromboangiitis obliterans (Buerger's disease), Behcet's disease, cutaneous vasculitides, Rickettsial vasculitis, disseminated intravascular coagulation, lymphoid interstitial pneumonia, eosinophilic granuloma of the lung, gastritis, chronic hepatitis, cirrhosis, Graves' disease, thyroiditis, hypothyroidism, psoriasis, Alzheimer's disease, allergic rhinitis, inflammatory dermatoses, cutaneous anaphylaxis reaction, and meningitis.

The term “comprising”, as used herein, means that various other compatible drugs and medicaments, as well as inert ingredients, can be conjointly employed in the pharmaceutical compositions and methods of this invention, as long as the defined pharmaceutically active compounds and carriers are used in the manner disclosed. The term “comprising” thus encompasses and includes the more restrictive terms “consisting of” and “consisting essentially of”.

The term “patient”, as used herein, is intended to encompass any mammal, animal or human, which may benefit from treatment with the compounds, compositions and methods of the present invention. “Treat,” “treating,” “treatment,” and “therapy” as used herein refer to any curative therapy, prophylactic therapy, ameliorative therapy and preventative therapy.

By “compatible” herein is meant that the components of the compositions which comprise the present invention are capable of being commingled without interacting in a manner which would substantially decrease the efficacy of the pharmaceutically active compound under ordinary use conditions.

The pharmaceutical compositions of the present invention comprise specifically-defined methimazole derivatives and tautomeric cyclic thiones, used in a safe and effective amount, together with a pharmaceutically-acceptable carrier.

The methimazole derivatives used in the compositions of the present invention are those having the following structural formulae:

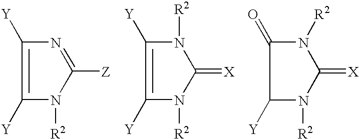

In these formulae, Y is selected from H, C1-C4 alkyl C1-C4 substituted alkyl, —NO₂, and the phenyl moiety: 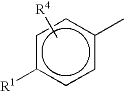
wherein no more than one Y group in said active compound may be the phenyl moiety; R¹ is selected from H, —OH, halogens (F, Cl, Br or I), C₁-C₄ alkyl, C₁-C₄ substituted alkyl, C₁-C₄ ester or C₁-C₄ substituted ester; R² is selected from H, C₁-C₄ alkyl or C₁-C₄ substituted alkyl; R³ is selected from H, C₁-C₄ alkyl, C₁-C₄ substituted alkyl or —CH₂Ph (wherein Ph is phenyl); R⁴ is selected from H, C₁-C₄ alkyl or C₁-C₄ substituted alkyl; X is selected from S or O; Z is selected from —SR³, —OR³, S(O)R³ or C₁-C₄ alkyl; and wherein at least two of the R² and R³ groups on said compound are C₁-C₄ alkyl when Y is not a phenyl moiety, and at least one Y is —NO₂ when Z is alkyl; together with a pharmaceutically-acceptable carrier.

Y is preferably H, the phenyl moiety or —NO₂, and is most preferably H or the phenyl moiety

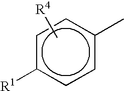

In the defined compounds, no more than one Y group may be the phenyl moiety. R¹ is selected from H, —OH, halogens (F, Cl, Br and I), C₁-C₄ alkyl, C₁-C₄ substituted alkyl, C₁-C₄ester and C₁-C₄ substituted ester; preferably R¹ is H, —OH, halogen, —OOC CH₂M (where M is H or a halogen); and is most preferably H. R² is selected from H, C₁-C₄ alkyl and C₁-C₄ substituted alkyl; preferably one or both of the R² groups is methyl. As used herein, “substituted alkyl” or “substituted ester” is intended to include alkyl, aryl or ester groups which are substituted in one or more places with hydroxyl or alkoxyl groups, carboxyl groups, halogens, nitro groups, amino or acylamino groups, and mixtures of those moieties. Preferred “substituted alkyl” groups are C₁-C₄ hydroxyl or alkoxyl groups, as well as groups substituted with halogens. The R³ groups in the above formulae are selected from H, C₁-C₄ alkyl, C₁-C₄ substituted alkyl and —CH₂Ph (wherein Ph is phenyl); in preferred compounds, R³ is H or C₁-C₄ alkyl; most preferably R³ is C₁-C₄ alkyl, particularly methyl. R⁴ is selected from H, C₁-C₄ alkyl and C₁-C₄ substituted alkyl, and preferably is H. X may be S or O, and is preferably S. Finally, Z is selected from C₁-C₄ alkyl, —SR³, —S(O)R³ and —OR³, is preferably —SR³, —OR³, and —S(O)R³; most preferably —SR³ and —OR³; and particularly —SR³. In the above formulae, at least two of the R² and R³ groups on the compound must be C₁-C₄ alkyl when Y is not a phenyl moiety. Further, at least one of the Y groups should be —NO₂, when Z is C₁-C₄ alkyl.

Compounds useful in the present invention include the tautomeric cyclic thiones, disclosed in Kjellin and Sandstrom, Acta Chemica Scandanavica 23: 2879-2887 (1969), incorporated herein by reference, having the formulae 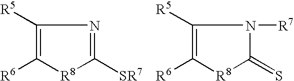
wherein R⁵, R⁶=CH₃, CH₃; Ph, H; H, Ph
R⁷=H, CH₃
R⁸=O, S, NH, NCH₃

Preferred compounds for use in the compositions of the present invention include those having the formulae:

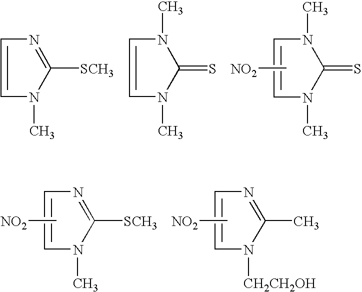

Another group of preferred compositions include those having the formulae: 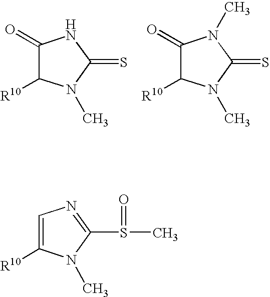
wherein R¹⁰ is selected from H. NO₂, Ph, 4-HOPh and 4-m-Ph (wherein m is F, Cl, Br, or I).

A particularly preferred subset of the pharmaceutical compounds defined herein are those wherein one of the Y groups is the phenyl moiety defined above. These compounds have the following formulae:

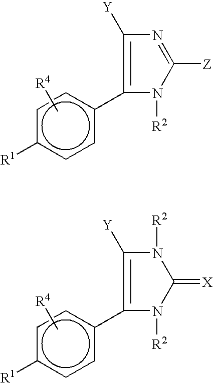

In these compounds, Y is selected from H, C₁-C₄ alkyl and C₁-C₄ substituted alkyl, and is preferably H. R¹ is selected from H, —OH, halogens (F, Cl, Br and I), C₁-C₄ alkyl, C₁-C₄ substituted alkyl, C₁-C₄ ester, and C₁-C₄ substituted ester, and is preferably H, —OH, halogen, —OOCCH₂M (where) M is H or a halogen), and is not preferably H. R² is selected from H, C₁-C₄ alkyl and C₁-C₄ substituted alkyl, and it is preferred that at least one of the R² groups be methyl. R³ is selected from H, C₁-C₄ alkyl, C₁-C₄ substituted alkyl, and —CH₂Ph; preferred R³ moieties are H and methyl. R⁴ is selected from H, C₁-C₄ alkyl and C₁-C₄ substituted alkyl, and is preferably H. X is selected from S and O, and is preferably S. Finally, the Z moiety is selected from —SR³ and —OR³, and is preferably —SR³. Particularly preferred compounds are those having the structural formulae:

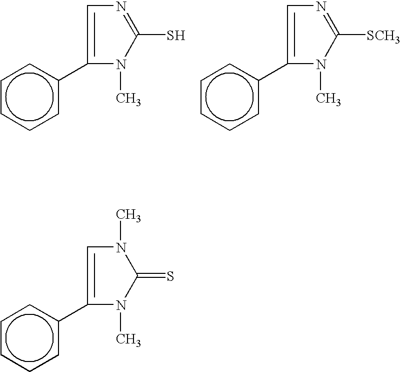

Other preferred compounds include: 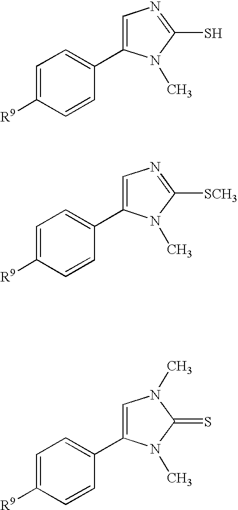
wherein R⁹ is selected from —OH, -M and —OOCCH₂ M; and M is selected from F, Cl, Br and I.

Most preferred is the compound having the structure given below.

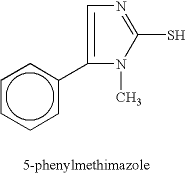

Mixtures of the pharmaceutically active compounds defined herein may also be used. The methimazole derivatives and tautomeric cyclic thiones described above can be synthesized using techniques well known to those skilled in the art. For example, the synthesis of several tautomeric cyclic thiones is described in Kjellin and Sandstrom, Acta Chemica Scandanavica 23: 2879.congruent.2887 (1969), incorporated herein by reference.

A representative methimazole derivative may be synthesized using the following procedure. Appropriately substituted analogs of acetaldehyde are brominated in the 2-position by treatment with bromine and UV light, followed by formation of the corresponding diethylacetal using absolute ethanol. The bromine is then displaced from this compound by treatment with anhydrous methylamine, or other suitable amine, in a sealed tube at about 120° for up to about 16 hours. Reaction of the resulting aminoacetal with potassium thiocyanate in the presence of hydrochloric acid, at steam bath temperatures overnight, provides the methimazole analogs.

TABLE 1
Structure of Compounds.

| Compounds | Imidazole | 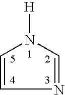 |
|---|---|---|
| #1 | 1-Methylimidazole-2- thiol (Methimzaole) C₄H₆N₂S; 1-Methyl-2- mercaptoimidazole (MMI) | 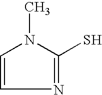 |
| #2 | 2-Methyl-5-nitro-1- imidazole ethanol (Metronidazole) C₆H₉N₃O₃; MW: 171.16 | 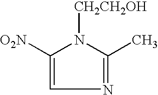 |
| #3 | 2-Mercaptoimidazole MW: 100.14 | 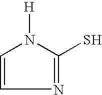 |
| #4 | 2-Mercaptobenzimida- zole MW: 150.20 | 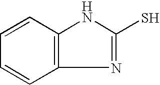 |
| #5 | 2-Mercapto-5- nitrobenzimidazole MW: 195.20 | 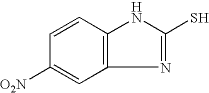 |
| #6 | 2-Mercapto-5- methylbenzimidazole MW: 164.23 | 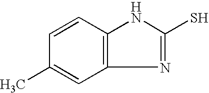 |
| #7 | S-Methylmethimazole C₅H₈N₂S; MW: 128.20 B.P. 48° @100u (liq.) | 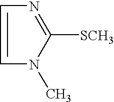 |
| #8 | N-Methylmethimazole C₅H₈N₂S; MW: 128.20 B.P. 188°-194° | 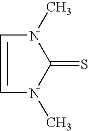 |
| #9 | 5-Methylmethimazole C₅H₈N₂S; MW: 128.20 B.P. 254°-255° | 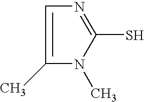 |
| #10 | 5-Phenylmethimazole C₁₀H₁₀N₂S; MW: 190.27 B.P. 168°-173° | 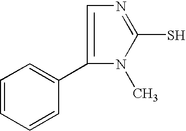 |
| #11 | 1-Methyl-2- Thiomethyl- 5(4)nitroimidazole | 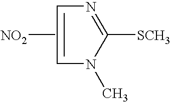 |

The pharmaceutical compositions of the present invention comprise a safe and effective amount of one or more of the methimazole derivatives or tautomeric cyclic thione compounds (i.e., the active compounds). Preferred compositions contain from about 0.01% to about 25% of the active compounds, with most preferred compositions containing from about 0.1% to about 10% of the active compounds. The pharmaceutical compositions of the present invention may be administered in any way conventionally known, for example, intraperitoneally, intravenously, intramuscularly, or topically, although oral administration is preferred. Preferred compositions are in unit dosage form, i.e., pharmaceutical compositions, which are available in a pre-measured form suitable for single dosage administration without requiring that the individual dosage be measured out by the user, for example, pills, tablets or ampules.

The pharmaceutical compositions of the present invention additionally include a pharmaceutically-acceptable carrier compatible with the methimazole derivatives or tautomeric cyclic thiones described above. In addition to the pharmaceutically-acceptable carrier, the pharmaceutical compositions may contain, at their art accepted levels, additional compatible ingredients, such as additional pharmaceutical actives, excipients, formulational aids (e.g., tabletting aids), colorants, flavorants, preservatives, solubilizing or dispersing agents, and other materials well known to those skilled in the art.

As used herein, the term “pharmaceutical carrier” denotes a solid or liquid filler, diluent or encapsulating substance. These materials are well known to those skilled in the pharmaceutical arts. Some examples of the substances which can serve as pharmaceutical carriers are sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; stearic acid; magnesium stearate; calcium sulfate; vegetable oils, such as peanut oil, cottonseed oil, sesame oil, olive oil, corn oil and oil of theobroma; polyols, such as propylene glycol, glycerine, sorbitol, mannitol, and polyethylene glycol; agar; alginic acid; pyrogen-free water; isotonic saline; and phosphate buffer solutions, as well as other non-toxic compatible substances used in pharmaceutical formulations. They may comprise liposomes or drug carriers made lipids or polymeric particles, including biodegradable polymers, or targeted delivery applications, e.g., coupling to antibodies. Wetting agents and lubricants, such as sodium lauryl sulfate, as well as coloring agents, flavoring agents, tableting agents, and preservatives, can also be present. Formulation of the components into pharmaceutical compositions is done using conventional techniques.

The pharmaceutical carrier employed in conjunction with the pharmaceutical compositions of the present invention is used at a concentration sufficient to provide a practical size-to-dosage relationship. Preferably, the pharmaceutical carrier comprises from about 75% to about 99.99%, preferably from about 90% to about 99.9%, by weight of the total pharmaceutical composition. The methimazole derivatives or tautomeric cyclic thiones defined in the present application may surprisingly be more soluble than methimazole in conventional carrier materials. This provides significant benefits in allowing greater flexibility in the formulation of pharmaceutical compositions containing those methimazole derivatives, and may allow the use of significantly lower doses of the active compound.

In its broadest aspects, methimazole derivatives of the present invention are administered in a dosage range of from about 0.001 to about 100 milligrams, preferably from about 0.05 to about 50 milligrams, per day. The pharmaceutical compositions of the present invention are administered such that appropriate levels of pharmaceutical active are achieved in the bloodstream. The precise dosage level required in a given case will depend upon, for example, the particular methimazole derivative used, the nature of the disease being treated, and the size, weight, age and physical condition of the patient.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids. It will be understood that, as used herein, the compounds referred to herein are meant to also include the pharmaceutically acceptable salts.

The ability of the therapeutic compounds of the present invention to inhibit the actions of VCAM-1 makes them useful for preventing or reversing the symptoms, disorders or diseases induced by the binding of VCAM-1 to its various-ligands (e.g., VLA-4). Thus, these compounds will inhibit rolling and cell adhesion processes including cell signaling, activation, migration, proliferation and differentiation. Accordingly, another aspect of the present invention provides a method for the treatment (including prevention, alleviation, amelioration or suppression) of diseases or disorders or symptoms mediated by VCAM-1 binding, and cell adhesion and activation, which comprises administering to a mammal an effective amount of a compound of the present invention. Such diseases, disorders, conditions or symptoms are for example (1) multiple sclerosis, (2) asthma, (3) allergic rhinitis, (4) allergic conjunctivitis, (5) inflammatory lung diseases, (6) rheumatoid arthritis, (7) septic arthritis, (8) type I or Type II diabetes and their macro- or microvascular complications, e.g. nephropathy, stroke, or myocardial infarct, (9) organ transplantation rejection, (10) restenosis, (11) autologous bone marrow transplantation, (12) inflammatory sequelae of viral infections, (13) myocarditis, (14) inflammatory bowel disease including ulcerative colitis and Crohn's disease, (15) certain types of toxic and immune-based nephritis, (16) contact dermal hypersensitivity, (17) psoriasis, (18) tumor metastasis, (19) thyroiditis, and (20) atherosclerosis.

The magnitude of prophylactic or therapeutic dose of the therapeutic compound of the present invention will, of course, vary with the nature of the severity of the condition to be treated and with the particular therapeutic compound of the present invention and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous or intraperitoneal administration is employed, a suitable dosage range is from about 0.001 mg to about 25 mg (preferably from 0.01 mg to about 1 mg) of a therapeutic compound of the present invention per kg of body weight per day and for cytoprotective use from about 0.1 mg to about 100 mg (preferably from about 1 mg to about 100 mg and more preferably from about 1 mg to about 10 mg) of the therapeutic compound of the present invention per kg of body weight per day.

In the case where an oral composition is employed, a suitable dosage range is, e.g. from about 0.01 mg to about 100 mg of the therapeutic compound of the present invention per kg of body weight per day, preferably from about 0.1 mg to about 10 mg per kg and for cytoprotective use from 0.1 mg to about 100 mg (preferably from about 1 mg to about 100 mg and more preferably from about 10 mg to about 100 mg) of a therapeutic compound of the present invention per kg of body weight per day.

For the treatment of diseases of the eye, ophthalmic preparations for ocular administration comprising 0.001-1% by weight solutions or suspensions of the therapeutic compound of the present invention in an acceptable ophthalmic formulation may be used.

Another aspect of the present invention provides pharmaceutical compositions which comprises a compound of the present invention and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of the present invention as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulisers. The compounds may also be delivered as powders, which may be formulated and the powder composition may be inhaled with the aid of an insufflation powder inhaler device. The preferred delivery systems for inhalation are metered dose inhalation (MDI) aerosol, which may be formulated as a suspension or solution of a compound of the present invention in suitable propellants, such as fluorocarbons or hydrocarbons and dry powder inhalation (DPI) aerosol, which may be formulated as a dry powder of a compound of the present invention with or without additional excipients.

Suitable topical formulations include transdermal devices, aerosols, creams, ointments, lotions, dusting powders, and the like.

In practical use, the compounds of the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques. In addition to the common dosage forms set out above, the therapeutic compound of the present invention may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

Compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention, such as methimazole derivatives and tautomeric cyclic thiones. When a compound of the present invention is used contemporaneously with one or more drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention I, either administered separately or in the same pharmaceutical compositions, include, but are not limited to: (a) VCAM-1 antagonists; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib; (h) inhibitors of phosphodiesterase type IV (PDE-IV); (i) antagonists of the chemokine receptors, especially CCR-1, CCR-2, and CCR-3; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), a-glucosidase inhibitors (acarbose) and glitazones (troglitazone, pioglitazone, englitazone, MCC-555, BRL49653 and the like); (1) preparations of type 1 interferon (e.g., beta-interferon and alpha-interferon); (m) anticholinergic agents such as muscarinic antagonists (ipratropium bromide); (n) other compounds such as 5-aminosalicylic acid and prodrugs thereof, antimetabolites such as azathioprine and 6-mercaptopurine, and cytotoxic cancer chemotherapeutic agents, (o) antibiotics.

The weight ratio of the therapeutic compound of the present invention to the second active ingredient may be varied and will depend upon the effective dose of each ingredient. Generally, an effective dose of each will be used. Thus, for example, when a therapeutic is combined with an NSAID the weight ratio of the compound of the therapeutic compound of the present invention to the NSAID will generally range from about 1000:1 to about 1:1000, preferably about 200:1 to about 1:200. Combinations of a therapeutic and other active ingredients will generally also be within the aforementioned range, but in each case, an effective dose of each active ingredient should be used.

The following examples are intended to illustrate the pharmaceutically active compounds, pharmaceutical compositions and methods of treatment of the present invention, but are not intended to be limiting thereof.

### EXAMPLES

The following examples are intended to illustrate the pharmaceutically active compounds, pharmaceutical compositions and methods of treatment of the present invention, but are not intended to be limiting thereof.

Pro-inflammatory cytokine (e.g. TNF-α)-induced expression of endothelial cell adhesion molecules (ECAMs) on the lumenal surface of the vascular endothelium, and a consequent increase in leukocyte adhesion, is a key aspect of pathological inflammation. The present invention provides for the use of methimazole derivatives and tautomeric cyclic thiones that (i) dramatically inhibit TNF-α-induced VCAM-1 mRNA and protein expression in human aortic endothelial cells (HAEC), has a relatively modest inhibitory effect on TNF-α induced E-selectin expression and have no effect on ICAM-1 expression; (ii) significantly reduce TNF-α induced monocytic (U937) cell adhesion to HAEC under in vitro flow conditions similar to that present in vivo; (iii) inhibit TNF-α induced interferon regulatory factor-1 (IRF-1) binding to VCAM-1 promoter and (iv) reduce TNF-α induced IRF-1 expression in HAEC cells. Combined, the results indicate that methimazole derivatives and tautomeric cyclic thiones can reduce TNF-α induced monocytic cell adhesion to HAEC predominantly by inhibiting VCAM-1 expression in an IRF-1 dependent manner.

Materials and Methods

Materials: Medium 199 (M199), RPMI 1640 (RPMI), and Hanks Balanced Salt Solution (HBSS) with Ca++ and Mg++ (HBSS+) all are from Biowhittaker (Walkersville, Md.). Delbecco's Phosphate Buffered Saline without Ca⁺⁺ or Mg⁺⁺ (PBS) is from KD Medical (Columbia, Md.). Heat inactivated defined fetal bovine serum (FBS) is from Hyclone Laboratories Inc. (Logan, Utah). L-glutamine, trypsin-versene, penicillin/streptomycin, and non-essential amino acid all are from Biosource International (Camarillo, Calif.). Endothelial growth factor is from Calbiochem (San Diego, Calif.). Bovine hypothalamus extract is from Pel-Freeze Biological Inc. (Rogers, Ark.). Gelatin, heparin, DMSO, BSA, O-phenylenediamine dihydrochloride (OPD) and phosphate citrate buffer tablets with sodium perborate are from Sigma Chemical Co. (St. Louis, Mo.). BSA is added to HBSS+ to generate a HBSS+, 0.5% BSA assay buffer. Recombinant human TNF-α is from Calbiochem. C-10 is synthesized as described by Ricerca (Cleveland, Ohio) (26). C-10 is prepared as 200 mM stock solution in DMSO.

Antibodies: Function blocking murine mAb HEL3/2 (anti-human E-selectin; IgG₁) is a generous gift from Dr. Raymond T. Camphausen (Wyeth Laboratories; Cambridge, Mass.). Function blocking murine mAb 51-10C9 (anti-human VCAM-1; IgG₁) is from BD Pharmingen (San Diego, Calif.). Murine mAb R6.5 (anti-human ICAM-1; IgG₂ₐ) is kindly provided by Dr. Robert Rothlein (Boehringer Ingelheim; Ridgefield, Conn.). Murine mAb TS 1/22 (anti-human LFA-1; IgG₁) is from Endogen (Woburn, Mass.). HRP conjugated goat F(ab′)₂ anti-mouse IgG polyclonal secondary antibody (Calbiochem) is used to detect the primary mAbs in the ELISA. The polyclonal antibodies to p50 (sc-1191), p65 (sc-109), p52 (sc-298), c-rel (sc-70), RelB (sc-226), IRF-1 (sc-1041×) are from Santa Cruz Biotechnology (Santa Cruz, Calif.).

Cell culture and treatment of HAEC: Human aortic endothelial cells (HAEC; Cambrex Bio Science Walkersville, Inc., Walkersville, Md.) are cultured in M199 supplemented with 8% FBS, 100 μg/ml heparin, 10 ng/ml endothelial growth factor, 100 μg/ml hypothalamus extract, 2 mM L-glutamine, 1% non-essential amino acids, 100 units/ml penicillin and 100 μg/ml streptomycin. HAEC are subcultured on gelatin pre-coated: 96-well tissue culture plates (Corning Incorporated; Corning, N.Y.) for viability assays and ELISA; 100-mm tissue culture dishes (BD Falcon; Franklin Lakes, N.J.) for Northern blot analyses, Western blot analyses and EMSA; 35-mm tissue culture dishes (Corning) for adhesion assays and on 24-well tissue culture plates for luciferase promoter assays (BD Falcon). All experiments are performed with confluent HAEC monolayers. Unless noted otherwise, HAEC are treated with 25 ng/ml TNF-α, in the absence or presence of C-10 or 0.25% DMSO (carrier control for C-10), for 2-24 hrs. The concentration of DMSO is held constant at 0.25% (unless indicated otherwise) for all of the C-10 conditions. We observed that treatment of HAEC with C-10, at the concentrations used in this study, has little to no effect on HAEC viability as determined by MTS assay (20) and visual inspection of the HAEC monolayers (data not shown).

U937 cells (American Type Culture Collection; Manassas, Va.) are cultured in RPMI supplemented with 8% FBS, 2 mM L-glutamine, 100 units/ml penicillin and 100 μg/ml streptomycin. For the adhesion assays, U937 cells are washed, resuspended to 1×10⁸ cells/ml in RPMI and held on ice (<4 hrs.) until the time they are used in the flow adhesion assay.

ELISA: ELISA is used to characterize the protein levels of ECAMs on HAEC in a manner similar to that described previously (20). HAEC are washed with cold HBSS+, fixed in 1% formaldehyde at 4° C. for 20 min., washed with cold HBSS+, and incubated in cold M199 containing 8% FBS. Unless otherwise noted, from this point on all antibody dilutions and washes are carried out with M199 containing 8% FBS. Murine mAbs (primary mAbs) to ECAMs are added (10 μg/ml) and the HAEC incubated at 4° C. for 20 min. Following the incubation, the wells are washed and a peroxidase conjugated polyclonal (secondary) antibody to mouse IgG is added (diluted 1:50). After a 20 min. incubation at 4° C., the wells are washed and treated with OPD dissolved in phosphate citrate buffer containing sodium perborate. Following a 10 min. incubation at room temperature, the absorbance of the fluid in each well is determined at 450 nm using a micro-well plate spectrophotometer (Molecular Devices; Sunnyvale, Calif.). In every experiment, each condition is run in triplicate wells.

RNA isolation and Northern blot analysis: Northern blot analysis is used to characterize the mRNA levels in a manner similar to that described previously (27, 29, 33). HAEC are washed with PBS and total RNA extracted using a commercial kit (RNeasy Mini Kit; Qiagen Inc.; Valencia, Calif.). 12 μg of total RNA per lane is resolved on 1% denaturing agarose gels containing 0.66M formaldehyde. Gels are capillary blotted on Nytran membranes (Schleicher and Schuell Inc.; Keene, N.H.), UV cross-linked, and used for hybridization. The probe for IRF-1 has been previously described (33). The G3PDH cDNA is from Clontech (Palo Alto, Calif.). Other probe sequences are synthesized by RT-PCR (33) using the following cDNA specific primers: VCAM-1, 5′GACTCCGTCTCATTGACTTGCAGCACCACAG 3′ and 5′ATACTCCCGCATCCTTCAACTGGGCCTTTCG 3′ (1876 bp); E-Selectin, 5′GTGCAGCCATTCCCCTGCTGGAGAGTTC 3′ and

5′GGGCCAGAGACCCGAGGAGAGTTATCTG 3′ (977 bp);
and
ICAM-1,
5′CTCAGGTATCCATCCATCCCAGAGAAGCCTTCC 3′
and
5′CCCTTGAGTTTTATGGCCTCCTCCTGAGCCTTC 3′ (1514 bp).

cDNAs are labeled with α-³²P-dCTP using the Ladderman Labeling Kit (Takara Biochemical Inc.; Berkeley, Calif.) (33). Northern blots are developed using a BAS 1500 Bioimaging Analyzer (Fuji Photo Film Co., Ltd. Medical Systems USA Inc.; Stanford, Calif.). Each experiment is replicated at least twice.

Nuclear Extracts and EMSA: Nuclear extracts are prepared from harvested HAEC using NE-PER® extraction reagents (Pierce Chemical Co.; Rockford, Ill.) in the presence of a protease inhibitor cocktail (PMSF, Leupeptin, Pepstatin-A). Oligonucleotides (Biosynthesis Inc.; Lewisville, Tex.) are annealed and precipitated double stranded oligonucleotides end labeled with γ-³²P-ATP using T4 polynucleotide kinase enzyme. Binding reactions (20 min., room temperature) included ³²P-labeled probe (activity 100,000 cpm), 3-6 μg HAEC nuclear extract, 1 μg poly(dI-dC), 1 mM DTT, 10% glycerol and 1× binding buffer. Binding buffer (10×) for NF-κB EMSA is 200 mM HEPES-KOH (pH7.9), 340 mM KCl, 50 mM MgCl₂, 5 mM EDTA (pH 8.0), 1% Triton X-100. Binding buffer (10×) for IRF-1 EMSA is 100 mM Tris-HCL (pH 7.5), 500 mM NaCl, 50 mM MgCl₂, 10 mM EDTA (pH 8.0). In competition studies, nuclear extracts are incubated with 100 fold molar excess of unlabeled double stranded oligonucleotide. In supershift studies, nuclear extracts are incubated with 2 μg of appropriate antibodies. After the incubations, reaction mixtures are electrophoresed (160V, room temperature) on 5% non-denaturing polyacrylamide gels containing 5% glycerol in 1×TBE (50 mM Tris, 50 mM boric acid, and 1 mM EDTA). Gels are dried and autoradiographed. Each experiment is replicated at least twice.

Flow adhesion assays: A parallel plate flow chamber (Glycotech; Rockville, Md.), similar to that described by McIntire, Smith and colleagues (34) is used in this study. Our particular set up has been described previously (20). A 35-mm tissue culture dish containing a confluent HAEC monolayer is loaded into the flow chamber. After a brief rinse, U937 cells (5*10⁵ cells/ml in assay buffer) are drawn over the HAEC monolayer at a shear stress of 1.8 dynes/cm². To determine U937 cell accumulation, the number of U937 cells adherent (either rolling or firmly adherent) to the HAEC monolayer in 8 different fields of view after 2.5 minutes of flow is determined, averaged and normalized to the area of the field of view to give the result for that particular run. Such an assay is done at least 3 times. In certain experiments, TNF-α activated HAEC, in the absence or presence of C-10, are treated with mAbs HEL3/2 or 51-10C9 (10 μg/ml) or a combination of HEL3/2 and 51-10C9. The mAb treated HAEC are then incubated at 37° C. for 15 min. prior to use in adhesion assays.

Dual Luciferase Assay: Four human VCAM-1 promoter deletion constructs of different length, −1641/+12, −288/+12, −228/+12, −85/+12 bp are amplified by PCR from human genomic DNA. Each upstream primer contained a restriction endonuclease Mlu I site located at the 5′-end of the primer. The downstream primer corresponding to the +12 end contained a restriction endonuclease Xho I site at the 5′-end. The PCR products are digested by Mlu I and Xho I (New England Biolabs Inc., Beverly, Mass.) and ligated into a similarly digested pGL3 basic luciferase reporter vector (Promega, Madison, Wis.). Cells are transfected for 24 hrs. with 400 ng of indicated constructs, or pGL3 basic luciferase reporter vector as control, using GeneJuice transfection reagent (Novagen Inc., Madison, Wis.). All cells are also transfected with phRL-TK (Int−) vector (Promega), which contains wildtype Renilla luciferase (Rluc) as an “internal” transfection control. Luciferase assays are conducted with the Dual-Luciferase Reporter Assay System (Promega) on a Lumat LB 9507 tube luminometer. In every experiment, each condition is run in triplicate wells. Each experiment is replicated at least twice.

Western blot analysis: Whole cell lysates are prepared in lysis buffer (150 mM NaCl, 1% IGEPAL CA-630, 50 mM Tris-HCL pH 8.0). 20 μg of lysates are then resolved on 4-12% Bis-Tris PAGE gels under denaturing conditions using the NuPAGE Bis-Tris System (Invitrogen; Carlsbad, Calif.). Proteins are transferred to nitrocellulose membranes, which are probed with IRF-1 antibody. Subsequent binding of HRP conjugated goat anti-rabbit Ab (sc-2054, Santa Cruz Biotechnology, Inc.) is detected using Lumigen PS-3 detection reagents (Amersham Pharmacia Biotech.; Piscataway, N.J.).

Statistics: A single factor ANOVA is used to assess the presence of statistical differences. If ANOVA indicated significant differences between conditions, a Bonferroni test is used for multiple pair-wise comparisons. Student's T-test is used to assess the statistical difference in luciferase promoter assays. P values <0.001 (for ELISA) and <0.05 (for adhesion and luciferase promoter assays) are considered statistically significant. Unless stated otherwise, all error bars represent standard deviation.

Results

Methimazole Derivatives and Tautomeric Cyclic Thiones Dramatically Inhibit TNF-α Induced VCAM-1 Expression have a Modest Effect on E-Selectin Expression, and has No Effect on ICAM-1 Expression

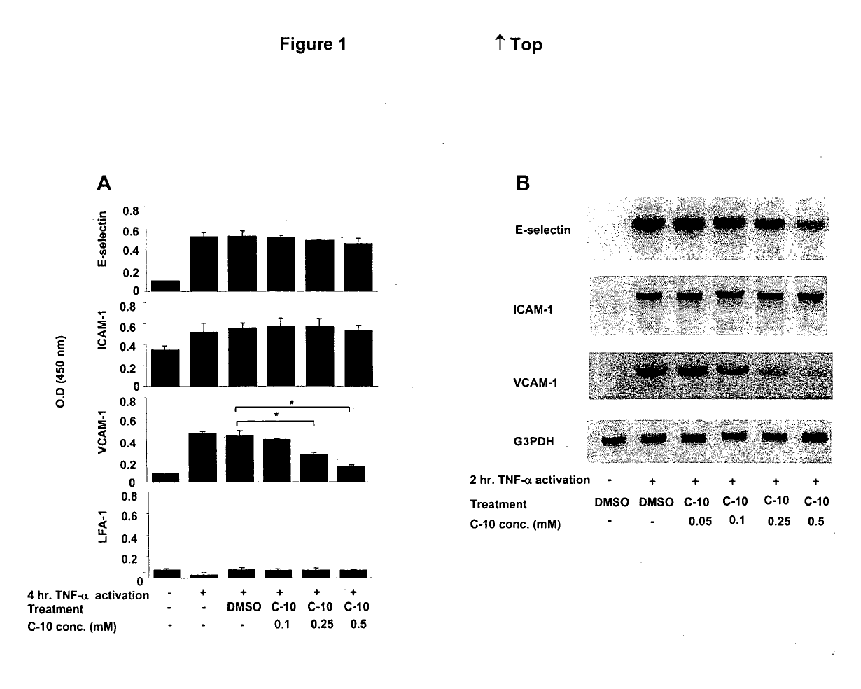

We sought to determine the effect of methimazole derivatives and tautomeric cyclic thiones on TNF-α induced ECAM expression using arterial endothelial cells, e.g., HAEC. Since the ECAM profile on 4 hr. and 24 hr. TNF-α treated endothelial cells is significantly different (6), we investigate the effect of C-10 on 4 hr. and 24 hr. TNF-α treatment of HAEC. We first determine the effect of C-10 on 4 hr. and 24 hr. TNF-α induced ECAM protein expression on HAEC. Unactivated HAEC does not appear to express E-selectin or VCAM-1 but did express ICAM-1 (FIG. 1A). 4 hr. treatment of HAEC with TNF-α induced E-selectin and VCAM-1 protein expression and significantly increased ICAM-1 protein expression (FIG. 1A). Treatment of HAEC with DMSO (carrier control) has little to no effect on 4 hr. TNF-α induced protein expression of E-selectin, ICAM-1 and VCAM-1 (FIG. 1A). In contrast, treatment of HAEC with C-10 significantly reduces 4 hr. TNF-α induced protein expression of VCAM-1 (FIG. 1A). This effect is observed with C-10 concentrations ≧0.25 mM (FIG. 1A). Treatment of HAEC with methimazole derivatives and tautomeric cyclic thiones has little, if any, effect on 4 hr. TNF-α induced protein expression of E-selectin and ICAM-1 (FIG. 1A). The expression at the protein level is paralleled at the mRNA level. Specifically, Northern blot analyses reveales that C-10 reduced, in a dose-dependent manner, TNF-α induced VCAM-1 mRNA expression, has a marginal effect on E-selectin mRNA expression and no effect on ICAM-1 mRNA expression (FIG. 1B).

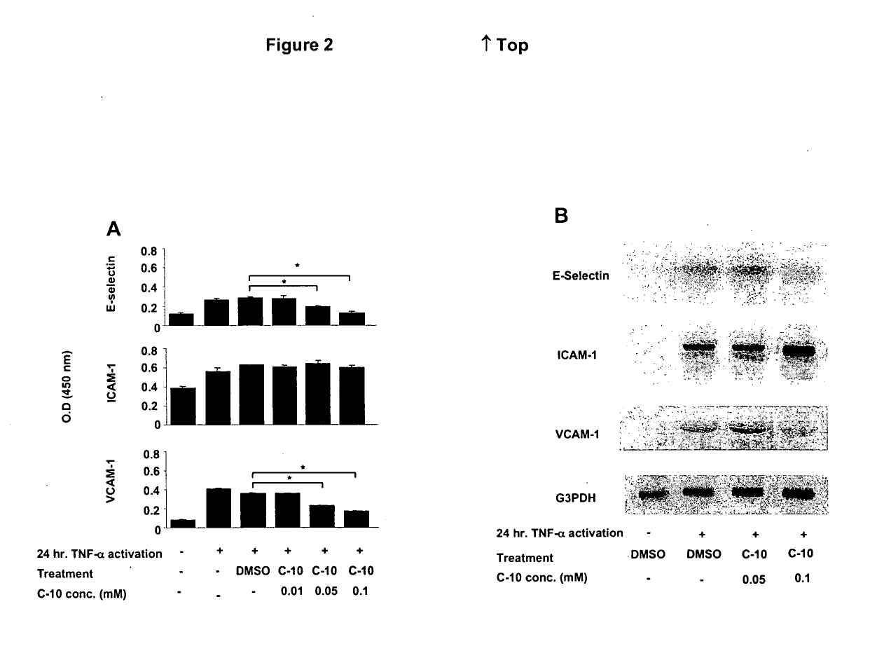

Similar results are observed at the 24 hr. time point with the exception that an inhibitory effect on E-selectin expression is also observed. HAEC treated with TNF-α for 24 hrs. expresses a level of E-selectin that is higher than the basal level (FIG. 2A) although distinctly less than the level seen at 4 hrs. post-TNF-α treatment (FIG. 1A). 24 hr. TNF-α activated HAEC also expresses elevated levels of ICAM-1 and VCAM-1 (relative to unactivated HAEC) (FIG. 2A). Treatment of HAEC with DMSO has little to no effect on the 24 hr. TNF-α induces expression of E-selectin, ICAM-1 and VCAM-1 (FIG. 2A). In contrast, treatment of HAEC with C-10 significantly reduces the 24 hr. TNF-α induces expression of E-selectin and VCAM-1 but has no effect on ICAM-1 expression (FIG. 2A). The effect is observed with C-10 concentrations ≧0.05 mM (FIG. 2A). Again, the expression at the protein level is paralleled at the mRNA level. Specifically, Northern blot analyses reveales that C-10 reduced, in a dose dependent manner, 24 hr. TNF-α induces E-selectin and VCAM-1 mRNA, while it has little to no effect on ICAM-1 mRNA expression (FIG. 2B).

The effect is not restricted to C10 but is exemplified by other methimazole derivatives or tautomeric cyclic thiones.

TABLE 1
Effect of different concentrations of MMI derivatives and
Tautomeric Cyclic Thiones on TNF-alpha-induced VCAM-1
RNA levels in aortic endothelial cells
% INHIBITION

| Compound | 10 :M | 100 :M | 5.0 mM |
|---|---|---|---|
| Methimazole | 0 | 0 | 20 ± 10 |
| 2-mercaptoimidazole | None | None | None |
| 2-mercaptobenzimidazole | 0 | 0 | 26 ± 10 |
| 2-mercapto-5- | ND | ND | 11 ± 7 |
| methylbenzimididazole | | | |
| N-methylmethimazole | 0 | 31 ± 10 | 47 ± 12 |
| 5-Phenylmethimazole | 75 ± 7 | 99 ± 4 | 99 ± 3 |
| 1-methyl-2-thiomethyl- | 34 ± 6 | 83 ± 10 | 92 ± 5 |
| 5(4)nitroimidazole | | | |

Values from three experiments in duplicate, mean ± SD.
ND is not done.
Bold values represent significant inhibition (P < 0.05 or better).

Methimazole Derivatives or Tautomeric Cyclic Thiones, e.g., C10, Inhibit Monocytic Cell Adhesion to TNF-α Activated HAEC

VCAM-1 has been shown to play a role in mononuclear leukocyte adhesion to vascular endothelium (35). This fact combined with our finding that C-10 significantly inhibits VCAM-1 protein expression (FIGS. 1A and 2A), led us to probe the effect of methimazole derivatives and tautomeric cyclic thiones on monocytic (U937) cell adhesion to 4 hr. and 24 hr. TNF-α activated HAEC. For this study we use an in vitro flow chamber that mimics flow conditions present in vivo and use C-10 concentrations (0.5 mM for 4 hr. and 0.1 mM for 24 hr.) that has maximal effects in our ELISA assays (FIGS. 1A and 2A).

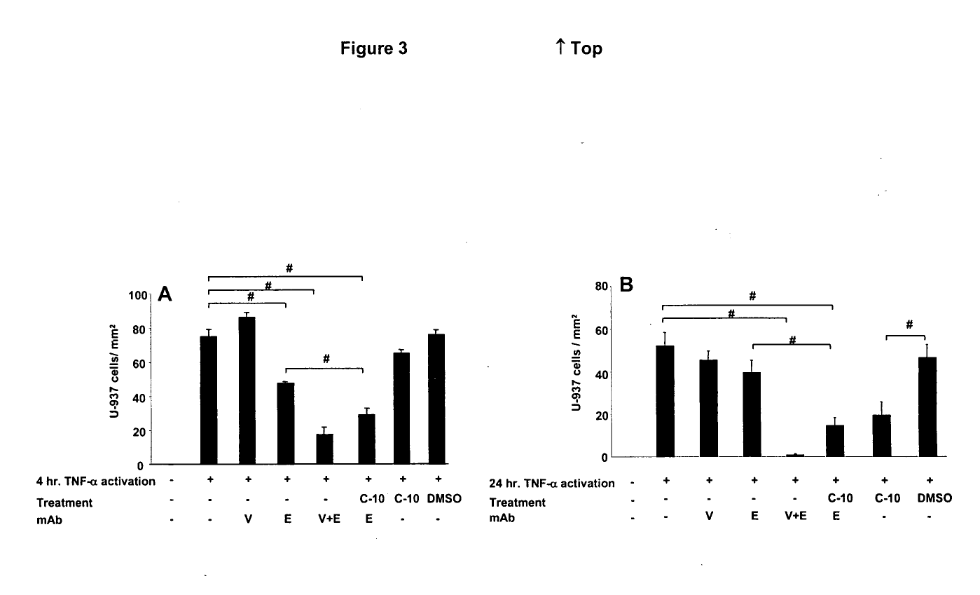

First, we use a mAb blocking approach to determine which ECAMs are involved in the adhesion of U937 cells to 4 hr. TNF-α activated HAEC. A significant number of U937 cells adhered to 4 hr. TNF-α activated HAEC (column 2; FIG. 3A) while very few, if any, U937 cells adhered to unactivated HAEC (column 1; FIG. 3A). U937 cell adhesion is unaffected by treatment of 4 hr. TNF-α activated HAEC with 51-10C9, a function blocking mAb to VCAM-1 (column 3 vs. column 2; FIG. 3A) and is partially reduced by treatment of 4 hr. TNF-α activated HAEC with HEL 3/2, a function blocking mAb to E-selectin (column 4 vs. column 2; FIG. 3A). Further reduction in U937 cell adhesion is seen upon treatment of 4 hr. TNF-α activated HAEC with a combination of 51-10C9 and HEL3/2 (column 5 vs. column 4; FIG. 3A). Combined these results suggest that U937 cell adhesion to 4 hr. TNF-α activated HAEC is mediated by both E-selectin and VCAM-1. This is consistent with other reports (18).

The combination of C-10 and the mAb to E-selectin (HEL3/2) significantly reduced 4 hr. TNF-α induced U937 cell adhesion (column 6 vs. column 2; FIG. 3A). In addition, the combination of C-10 and the mAb to E-selectin significantly reduced the 4 hr. TNF-α induced U937 cell adhesion relative to treatment with the mAb to E-selectin alone (column 6 vs. column 4; FIG. 3A). Treatment of HAEC with C-10 alone has little effect on the 4 hr. TNF-α induced U937 cell adhesion compared to treatment with DMSO (column 7 vs. column 8; FIG. 3A). Combined, this data demonstrates that C-10 has a modest effect on 4 hr. TNF-α induced U937 cell adhesion to HAEC, consistent with the mAb data above showing inhibition required mAbs to E-selectin and VCAM-1 and with evidence in FIG. 1 showing that at 4 hrs. C-10 has a selective effect on VCAM-1 mRNA and protein expression.

A more dramatic effect is observed at the 24 hr. TNF-α activation time point (FIG. 3B). A significant number of U937 cells adhered to 24 hr. TNF-α activated HAEC (column 2; FIG. 3B) while very few, if any, U937 cells adhered to unactivated HAEC (column 1; FIG. 3B). The U937 cell adhesion to 24 hr. TNF-α activated HAEC is dependent on both E-selectin and VCAM-1 (column 5 vs. columns 2,3,4; FIG. 3B). The combination of C-10 and the mAb to E-selectin (HEL3/2) significantly reduced 24 hr. TNF-α induced U937 cell adhesion (column 6 vs. column 2; FIG. 3B). In addition, the combination of C-10 and the mAb to E-selectin significantly reduced the 24 hr. TNF-α induced U937 cell adhesion relative to treatment with the mAb to E-selectin alone (column 6 vs. column 4; FIG. 3B). Treatment of HAEC with C-10 alone also significantly reduced the 24 hr. TNF-α induced U937 cell adhesion relative to treatment with DMSO (column 7 vs. column 8; FIG. 3B). Combined, the above results clearly demonstrate that C-10 significantly reduces long term (24 hr.) TNF-α induced U937 cell adhesion to HAEC.

Methimazole Derivatives or Tautomeric Cyclic Thiones, e.g., C10 Effect VCAM-1 Gene Transcription

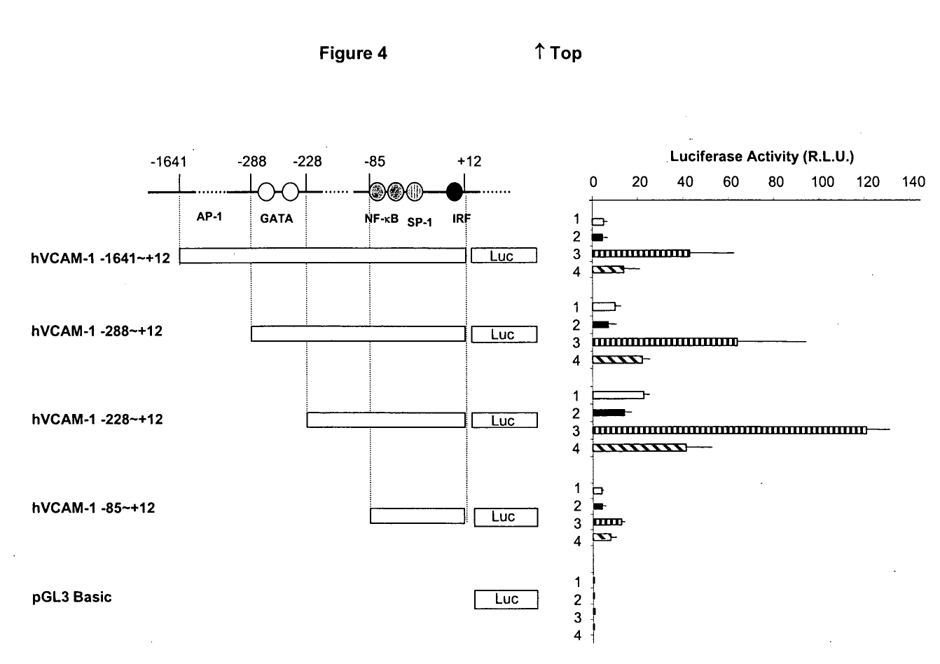

It is evident from above that methimazole derivatives, under both conditions tested, inhibit TNF-α induced VCAM-1 expression (FIGS. 1 and 2). To probe whether methimazole derivatives act transcriptionally to inhibit TNF-α induced VCAM-1 gene expression and also to get an idea of the molecular mechanism of C-10's inhibitory action, we conducted VCAM-1 promoter reporter assays. The locations of the binding sites for various transcription factors known to play a role in TNF-α-induced human VCAM-1 expression, NF-κB, AP-1, SP-1, IRF-1 and GATA, lie between −1641 and +12 as noted in FIG. 4. (8-11). Four truncations of the VCAM-1 transcriptional regulatory element are created (−1641/+12, −288/+12, −228/+12 and −85/+12 bp constructs) in an attempt to grossly separate their activities (FIG. 4). These are then inserted into a luciferase reporter plasmid and transfected into HAEC.

TNF-α treatment induces an increase in promoter activities of each of the four constructs (FIG. 4). C-10 treatment inhibits the TNF-α induced activities of all four constructs (FIG. 4) in the absence of a consistent significant effect on basal promoter activity (FIG. 4). Note that although the TNF-α-induced increase in promoter activity decreases between the −228/+12 and −85/+12 bp constructs (FIG. 4), an inhibitory effect of C-10 is observed with the 85/+12 bp construct (FIG. 4). Combined, these data clearly demonstrate that C-10 affects VCAM-1 gene transcription and show that methimazole derivatives and tautomeric cyclic thiones acts on a transcriptional regulatory event that occurs within −85/+12 bp of the VCAM-1 promoter.

The effect is not restricted to C10 but is exemplified by other methimazole derivatives or tautomeric cyclic thiones.

TABLE 2
Effect of different concentrations of MMI derivatives and
Tautomeric Cyclic Thiones on TNF-alpha-induced VCAM-1
promoter activity using hVCAM-1( −228 to +12)-luciferase
construct activity
% INHIBITION

| Compound | 10 :M | 100 :M | 5.0 mM |
|---|---|---|---|
| Methimazole | 0 | 0 | 0 |
| 2-mercaptoimidazole | 0 | 0 | 0 |
| 2-mercaptobenzimidazole | 0 | 0 | 30 ± 4 |
| N-methylmethimazole | 6 ± 2 | 44 ± 11 | 31 ± 3 |
| 5-methylmethimazole | 0 | 0 | 14 ± 8 |
| 5-Phenylmethimazole | 31 ± 5 | 61 ± 3 | 72 ± 3 |
| 1-methyl-2-thiomethyl- | 29 ± 6 | 54 ± 10 | 58 ± 6 |
| 5(4)nitroimidazole | | | |

Values from three experiments in duplicate, mean ± SD.
ND is not done.
Bold values represent significant inhibition (P < 0.05 or better).

Methimazole Derivatives or Tautomeric Cyclic Thiones, e.g., C10, do Not Affect TNF-α Induced NF-κB Binding Activity to VCAM-1 Promoter

The binding sites for NF-κB, in the VCAM-1 promoter, are located within −85/+12 bp (FIG. 4) (8, 9, 11). To determine if the inhibitory effect of C-10 on TNF-α induced VCAM-1 expression is a consequence of C-10 inhibition of TNF-α induced NF-κB activity we conduct EMSA. EMSA are performed with ³²P-labeled NF-κB probe and 6 μg of nuclear extract prepared from HAEC treated with or without TNF-α, in the absence or presence of C-10 (results not shown). Upon 2 hr. TNF-α treatment a complex is induced, which is prominent by comparison to the control, no TNF-α treatment. Competition with 100-fold molar excess of unlabeled NF-κB probe eliminated the TNF-α induced complex formation. Addition of 0.5 mM C-10 or DMSO has no effect on TNF-α dependent complex formation (data not shown). To identify the components of the complex, we performed supershift studies using antibodies to various NF-κB subunits. Antibodies directed against p50 and p65 subunits of NF-κB supershifted the TNF-α induced complex, whereas antibodies directed against p52, c-rel and rel-B did not (data not shown). Addition of 0.5 mM C-10 also has no effect in supershift studies (data not shown). Note that DMSO or C-10 treatment alone also has no effect on unactivated HAEC. Without wishing to be bound by theory in any way, the data strongly suggest that the mechanism of methimazole derivatives and tautomeric cyclic thiones inhibition of ECAM expression is not via inhibition of NF-κB activation and binding to VCAM-1 promoter.

Methimazole Derivatives or Tautomeric Cyclic Thiones, e.g., C10, Inhibit TNF-α Induced IRF-1 Binding Activity to VCAM-1 Promoter

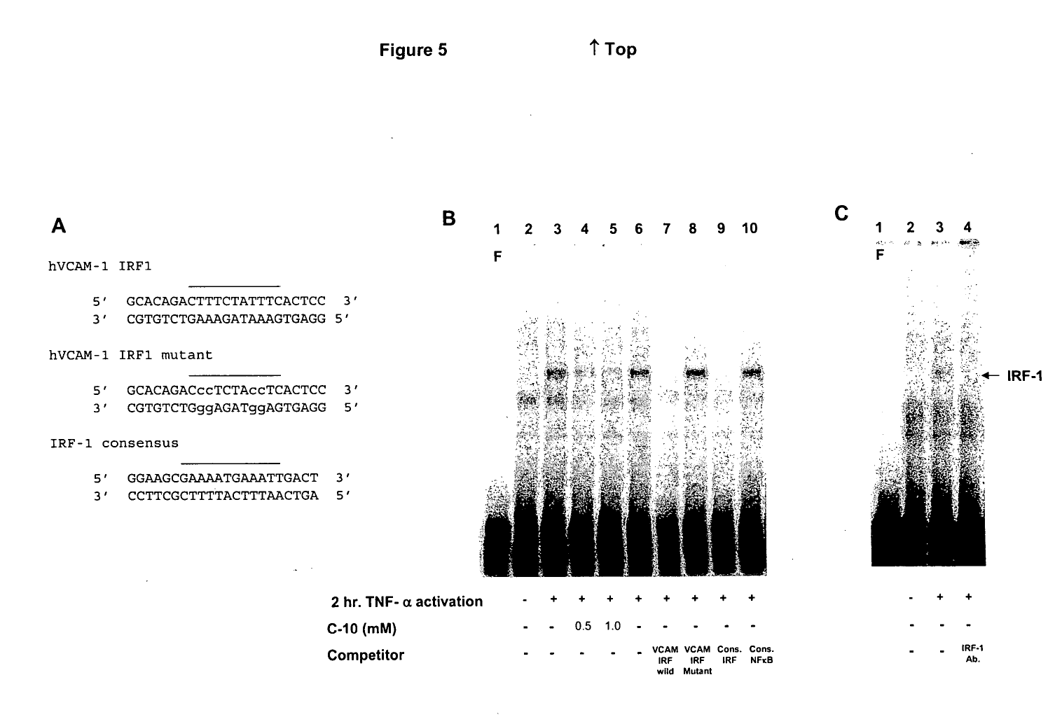

Because luciferase reporter assays suggestes that methimazole derivatives and tautomeric cyclic thiones affect a transcriptional regulatory event that occurs within −85/+12 bp in the VCAM-1 promoter (FIG. 4) and because EMSA demonstrated that C-10 does not affect TNF-α induced NF-κB activation, we considered the possibility that methimazole derivatives and tautomeric cyclic thiones might act at a different downstream site. The binding site for IRF-1 is located downstream of NF-κB binding sites within the −85/+12 bp, −11 to −1 bp, of the VCAM-1 promoter (8, 10, 11). To determine if the inhibitory effect of methimazole derivatives and tautomeric cyclic thiones on TNF-α induced VCAM-1 expression is a consequence of methimazole derivatives and tautomeric cyclic thiones inhibition of TNF-α induced IRF-1 activity we conduct EMSA. EMSA are performed with ³²P-labeled IRF-1 probe (FIG. 5A) and 3 μg of nuclear extract prepared from HAEC treated with or without TNF-α, in the absence or presence of C-10 (FIG. 5B). Upon 2 hr. TNF-α treatment, a complex is induced (lane 3; FIG. 5B) which is extremely prominent by comparison with control, no TNF-α treatment (lane 3 vs. lane 2; FIG. 5B). Addition of 0.5 mM and 1 mM C-10 inhibits the formation of TNF-α induced complex (lanes 4 and 5 vs. lane 3; FIG. 5B). Note, DMSO has no effect on TNF-α dependent complex formation (lane 6 vs. lane 3; FIG. 5B). Competition with 100-fold molar excess of unlabeled VCAM-1 IRF-1 probe (lane 7; FIG. 5B) or consensus IRF-1 probe (lane 9; FIG. 5B) eliminates the TNF-α induced complex formation. However, competition with 100-fold molar excess of unlabeled VCAM-1 IRF-1 mutant probe (lane 8; FIG. 5B) or consensus NF-κB probe (lane 10; FIG. 5B) did not affect the TNF-α induced complex formation. In supershift assays (FIG. 5C), we found that an antibody directed against IRF-1 supershifts the TNF-α induced complex (lane 4 vs. lane 3; FIG. 5C). Combined, these data demonstrate that methimazole derivatives and tautomeric cyclic thiones affect IRF-1 binding activity to VCAM-1 promoter and strongly suggest that methimazole derivatives and tautomeric cyclic thiones inhibit VCAM-1 expression in an IRF-1 dependent manner.

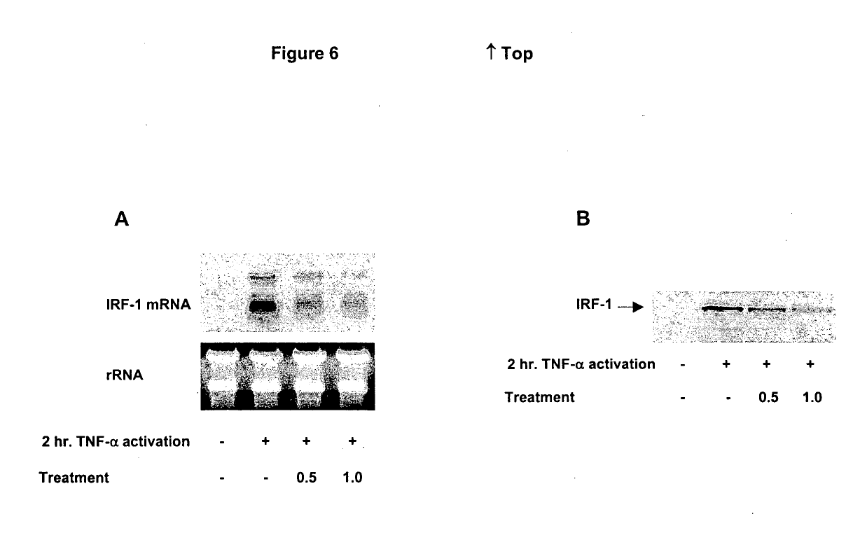

To further probe and determine the mechanism by which methimazole derivatives and tautomeric cyclic thiones inhibit TNF-α induced IRF-1 binding to VCAM-1 promoter (FIG. 5B), we investigate the effects of methimazole derivatives and tautomeric cyclic thiones on TNF-α induced IRF-1 protein and mRNA expression. Unactivated HAEC did not appear to express IRF-1 mRNA (FIG. 6A). 2 hr. treatment of HAEC with TNF-α induced IRF-1 mRNA (FIG. 6A). Addition of 0.5 mM or 1 mM C-10 reduces the TNF-α induced IRF-1 mRNA expression in HAEC (FIG. 6A). The expression at the mRNA level is paralled at the protein level. Specifically, C-10 treatment of HAEC reduced TNF-α induced IRF-1 protein expression (FIG. 6B). Combined, the data presented in this section demonstrates that methimazole derivatives and tautomeric cyclic thiones reduce TNF-α induced IRF-1 protein and mRNA expression.

These effects are not restricted to C10 but is exemplified by other methimazole derivatives or tautomeric cyclic thiones.

TABLE 3
Effect of different concentrations of MMI derivatives and
Tautomeric Cyclic Thiones on TNF-alpha-induced IRF-1 RNA levels
% INIHIBITION

| Compound | 10 :M | 100 :M | 5 mM |
|---|---|---|---|
| Methimazole | 0 | 0 | 27 ± 12 |
| 2-mercaptoimidazole | 0 | 0 | 0 |
| N-methylmethimazole | 0 | 34 ± 15 | 75 ± 14 |
| 5-Phenylmethimazole | 65 ± 16 | 90 ± 17 | 95 ± 15 |
| 1-methyl-2-thiomethyl- | 26 ± 12 | 64 ± 16 | 95 ± 12 |
| 5(4)nitroimidazole | | | |

Values from two experiments in duplicate, mean ± SD.
Bold values represent significant inhibition (P < 0.05 or better).

TABLE 4
Effect of different concentrations of MMI derivatives and Tautomeric
Cyclic Thiones on TNF-alpha-induced IRF-1 Protein levels
% INHIBITION

| Compound | 10 :M | 100 :M | 5 mM |
|---|---|---|---|
| Methimazole | 0 | 0 | 17 ± 6 |
| 5-Phenylmethimazole | 76 ± 12 | 95 ± 12 | 95 ± 5 |
| 1-methyl-2-thiomethyl- | 46 ± 15 | 69 ± 10 | 95 ± 12 |
| 5(4)nitroimidazole | | | |

Values from two experiments in duplicate, mean ± SD.
Bold values represent significant inhibition (P < 0.05 or better).

Discussion. In this study, we have found that C-10, a phenyl derivative of methimazole (a compound commonly used to treat autoimmune diseases, e.g. Graves' disease), has novel anti-inflammatory properties. Specifically, methimazole derivatives and tautomeric cyclic thiones dramatically inhibit TNF-α induced VCAM-1 mRNA and protein expression, have a relatively modest inhibitory effect on E-selectin expression and have no effect on ICAM-1 expression. We show that the effect on VCAM-1 inhibition is transcriptional and that methimazole derivatives and tautomeric cyclic thiones significantly reduce TNF-α induced monocytic cell adhesion to HAEC under in vitro flow conditions similar to that present in vivo.

Several current and potential anti-inflammatory agents diminish leukocyte adhesion by inhibiting cytokine induced ECAM expression at the transcription level (5). Not all of these compounds exert the same effect on cytokine induced ECAM expression. For example, lactacystin, can reduce the cytokine induced expression of E-selectin, ICAM-1 and VCAM-1 (20) while other compounds appear to be selective for one particular ECAM (e.g. VCAM-1; (19, 36)). Since the leukocyte adhesion cascade is documented to have receptor—ligand functional overlap (e.g. both E-selectin and VCAM-1 have been shown to support tethering and rolling of lymphocytes (37, 38)) compounds that suppress the expression of several of the ECAMs may be more effective at blocking leukocyte adhesion in a variety of inflammation settings. Specifically, methimazole derivatives and tautomeric cyclic thiones exert a greater inhibitory effect on TNF-α induced VCAM-1 expression as compared to the effect on E-selectin and ICAM-1 and can diminish monocytic cell adhesion to the endothelium under flow.

The present invention provides that methimazole derivatives and tautomeric cyclic thiones inhibit TNF-α induced VCAM-1 expression in a NF-κB independent and an IRF-1 dependent manner. Further, methimazole derivatives and tautomeric cyclic thiones can also be used tools to probe the role of IRF-1 in gene regulation.

The increase in TNF-α-induced VCAM-1 promoter activity is not significantly altered by deleting the AP-1 site between −1641 and −288 bp (FIG. 4). This is consistent with previous promoter assays (8) and a report indicating that the AP-1 effect is mediated through the NF-κB element (41). The deletion of −288 to −228 bp with no clear loss of TNF-α induced promoter activity (FIG. 4) is noteworthy given previous studies with other endothelial cell types (8, 42). Interestingly, the TNF-α-induced increase in promoter activity decreased between the −228/+12 and −85/+12 bp constructs, despite the fact the NF-κB elements are intact in the −85/+12 bp constructs (FIG. 4).

In conclusion, the present invention shows that methimazole derivatives and tautomeric cyclic thiones exhibit anti-inflammation properties. Specifically, phenyl methimazole (i) dramatically inhibits TNF-α induced VCAM-1 expression, has a modest inhibitory effect on E-selectin expression and has no effect on ICAM-1 expression on HAEC; (ii) significantly reduces TNF-α induced monocytic (U937) cell adhesion to HAEC under in vitro flow conditions similar to that present in vivo; (iii) inhibits TNF-α induced IRF-1 binding activity to VCAM-1 promoter and (iv) reduces TNF-α induced IRF-1 expression in HAEC. Thus, methimazole derivatives and tautomeric cyclic thiones can be used as a therapeutic for the treatment of pathological inflammation, in particular diseases involving VCAM-1 (e.g., atherosclerosis and inflammatory bowel disease).

Pharmaceutical Compositions of the Present Invention

For the treatment of cell adhesion and inflammation disorders, pharmaceutical compositions in dosage unit form comprise an amount of composition which provides from about 0.05 to about 60 milligrams, preferably from about 0.05 to about 20 milligrams, of active compound per day. Useful pharmaceutical formulations for administration of the active compounds of this invention may be illustrated below. They are made using conventional techniques.

CAPSULES

| Active ingredient | 0.05 to 20 | mg |
|---|---|---|
| Lactose | 20-100 | mg |
| Corn Starch U.S.P. | 20-100 | mg |
| Aerosolized silica gel | 2-4 | mg |
| Magnesium stearate | 1-2 | mg |

TABLETS

| | | |
|---|---|---|
| Active ingredient | 0.05 to 20 | mg |
| Microcrystalline cellulose | 50 | mg |
| Corn Starch U.S.P. | 80 | mg |
| Lactose U.S.P. | 50 | mg |
| Magnesium stearate U.S.P. | 1-2 | mg |

This tablet can be sugar coated according to conventional art practices. Colors may be added to the coating.

CHEWABLE TABLETS

| Active ingredient | 0.05 to 20 | mg |
|---|---|---|
| Mannitol, N.F. | 100 | mg |
| Flavor | 1 | mg |
| Magnesium stearate U.S.P. | 2 | mg |

SUPPOSITORIES

| | | |
|---|---|---|
| Active ingredient | 0.05 to 20 | mg |
| Suppository base | 1900 | mg |

| | |
|---|---|
| Dimethyl sulfoxide | 0.1 to 3% |

LIQUID

| | | |
|---|---|---|
| Active ingredient | 2.0 | percent |
| Polyethylene glycol 300, N.F. | 10.0 | percent |
| Glycerin | 5.0 | percent |
| Sodium bisulfite | 0.02 | percent |
| Sorbitol solution 70%, U.S.P. | 50 | percent |
| Methylparaben, U.S.P. | 0.1 | percent |
| Propylparaben, U.S.P. | 0.2 | percent |
| Distilled water, U.S.P. (q.s.) | 100.0 | cc |

| | |
|---|---|
| Dimethyl sulfoxide | 0.1 to 3% |

INJECTABLE

| | | |
|---|---|---|
| Active ingredient | 0.05 to 60 | mg |
| Polyethylene glycol 600 | 1.0 | cc |
| Sodium bisulfite, U.S.P. | 0.4 | mg |
| Water for injection, U.S.P. (q.s.) | 2.0 | cc |

| | |
|---|---|
| Dimethyl sulfoxide | 0.1 to 3% |

In addition, information regarding procedural or other details supplementary to those set forth herein is described in cited references specifically incorporated herein by reference.

It would be obvious to those skilled in the art that modifications or variations may be made to the preferred embodiment described herein without departing from the novel teachings of the present invention. All such modifications and variations are intended to be incorporated herein and within the scope of the claims.

### REFERENCES

1. Cybulsky, M. I., and M. A. Gimbrone, Jr. 1991. Endothelial expression of a mononuclear leukocyte adhesion molecule during atherogenesis. Science 251:788-791.
2. Schurmann, G. M., A. E. Bishop, P. Facer, M. Vecchio, J. C. Lee, D. S. Rampton, and J. M. Polak. 1995. Increased expression of cell adhesion molecule P-selectin in active inflammatory bowel disease. Gut 36:411-418.
3. Luscinskas, F. W., and M. A. Gimbrone. 1996. Endothelial-dependent mechanisms in chronic inflammatory leukocyte recruitment. Annu. Rev. Med. 47:413-421.
4. Soriano, A., A. Salas, M. Sans, M. Gironella, M. Elena, D.C. Anderson, J. M. Pique, and J. Panes. 2000. VCAM-1, but not ICAM-1 or MAdCAM-1, immunoblockade ameliorates DSS-induced colitis in mice. Lab. Invest. 80:1541-1551.
5. Panes, J., M. Perry, and D. N. Granger. 1999. Leukocyte-endothelial cell adhesion: avenues for therapeutic intervention. Br J Pharmacol 126:537-550.
6. Bevilacqua, M. P. 1993. Endothelial-leukocyte adhesion molecules. Annu. Rev. Immunol. 11:767-804.
7. Schindler, U., and V. R. Baichwal. 1994. Three NF-kappa B binding sites in the human E-selectin gene required for maximal tumor necrosis factor alpha-induced expression. Mol Cell Biol 14:5820-5831.
8. Neish, A. S., A. J. Williams, H. J. Palmer, M. Z. Whitley, and T. Collins. 1992. Functional analysis of the human vascular cell adhesion molecule 1 promoter. J Exp Med 176:1583-1593.
9. Neish, A. S., L. M. Khachigian, A. Park, V. R. Baichwal, and T. Collins. 1995. Sp1 is a component of the cytokine-inducible enhancer in the promoter of vascular cell adhesion molecule-1. J Biol Chem 270:28903-28909.
10. Neish, A. S., M. A. Read, D. Thanos, R. Pine, T. Maniatis, and T. Collins. 1995. Endothelial interferon regulatory factor 1 cooperates with NF-kappa B as a transcriptional activator of vascular cell adhesion molecule 1. Mol Cell Biol 15:2558-2569.
11. Iademarco, M. F., J. J. McQuillan, G. D. Rosen, and D.C. Dean. 1992. Characterization of the promoter for vascular cell adhesion molecule-1 (VCAM-1). J Biol Chem 267:16323-16329.
12. Ledebur, H. C., and T. P. Parks. 1995. Transcriptional regulation of the intercellular adhesion molecule-1 gene by inflammatory cytokines in human endothelial cells. Essential roles of a variant NF-kappa B site and p65 homodimers. J Biol Chem 270:933-943.
13. Munoz, C., M. C. Castellanos, A. Alfranca, A. Vara, M. A. Esteban, J. M. Redondo, and M. O. de Landazuri. 1996. Transcriptional up-regulation of intracellular adhesion molecule-1 in human endothelial cells by the antioxidant pyrrolidine dithiocarbamate involves the activation of activating protein-1. J Immunol 157:3587-3597.
14. May, M. J., and S. Ghosh. 1998. Signal transduction through NF-kappa B. Immunol Today 19:80-88.
15. Conner, E. M., S. Brand, J. M. Davis, F. S. Laroux, V. J. Palombella, J. W. Fuseler, D. Y. Kang, R. E. Wolf, and M. B. Grisham. 1997. Proteasome inhibition attenuates nitric oxide synthase expression, VCAM-1 transcription and the development of chronic colitis. J Pharmacol Exp Ther 282:1615-1622.
16. Pierce, J. W., M. A. Read, H. Ding, F. W. Luscinskas, and T. Collins. 1996. Salicylates inhibit I kappa B-alpha phosphorylation, endothelial-leukocyte adhesion molecule expression, and neutrophil transmigration. J Immunol 156:3961-3969.
17. Pierce, J. W., R. Schoenleber, G. Jesmok, J. Best, S. A. Moore, T. Collins, and M. E. Gerritsen. 1997. Novel inhibitors of cytokine-induced IkBa phosphorylation and endothelial cell adhesion molecule expression show anti-inflammatory effects in vivo. J. Biol. Chem. 272:21096-21103.
18. Weber, C., W. Erl, A. Pietsch, and P. C. Weber. 1995. Aspirin inhibits nuclear factor-kappa B mobilization and monocyte adhesion in stimulated human endothelial cells. Circulation 91:1914-1917.
19. Umetani, M., H. Nakao, T. Doi, A. Iwasaki, M. Ohtaka, T. Nagoya, C. Mataki, T. Hamakubo, and T. Kodama. 2000. A novel cell adhesion inhibitor, K-7174, reduces the endothelial VCAM-1 induction by inflammatory cytokines, acting through the regulation of GATA. Biochem Biophys Res Commun 272:370-374.
20. Dagia, N. M., and D. J. Goetz. 2003. A proteasome inhibitor reduces concurrent, sequential and long term IL-1β and TNF-α induced endothelial cell adhesion molecule expression and adhesion. Am. J. Phys. 285:C813-C822.
21. Cooper, D. S. 1984. Antithyroid drugs. N Engl J Med 311:1353-1362.
22. Elias, A. N., R. J. Barr, M. K. Rohan, and K. Dangaran. 1995. Effect of orally administered antithyroid thioureylenes on PCNA and P53 expression in psoriatic lesions. Int J Dermatol 34:280-283.
23. Chan, C. C., I. Gery, L. D. Kohn, R. B. Nussenblatt, E. Mozes, and D. S. Singer. 1995. Periocular inflammation in mice with experimental systemic lupus erythematosus. A new experimental blepharitis and its modulation. J Immunol 154:4830-4835.
24. Davies, T. F., I. Weiss, and M. A. Gerber. 1984. Influence of methimazole on murine thyroiditis. Evidence for immunosuppression in vivo. J Clin Invest 73:397-404.
25. Wang, P., S. H. Sun, P. B. Silver, C. C. Chan, R. K. Agarwal, B. Wiggert, L. D. Kohn, G. A. Jamieson, Jr., and R. R. Caspi. 2003. Methimazole protects from experimental autoimmune uveitis by inhibiting antigen presenting cell function and reducing antigen priming. J Leukoc Biol 73:57-64.
26. Kohn, L. D., R. W. J. Curley, and J. M. Rice. 2002. Methimazole derivatives and tautomeric cyclic thiones to treat autoimmune diseases. U.S. Pat. No. 6,365,616.
27. Saji, M., J. Moriarty, T. Ban, D. S. Singer, and L. D. Kohn. 1992. Major histocompatibility complex class I gene expression in rat thyroid cells is regulated by hormones, methimazole, and iodide as well as interferon. J Clin Endocrinol Metab 75:871-878.
28. Singer, D. S., L. D. Kohn, E. Mozes, M. Saji, J. Weissman, G. Napolitano, and F. D. Ledley. 1996. Methods for assessing the ability of a candidate drug to suppress MHC class I expression. U.S. Pat. No. 5,556,754.
29. Montani, V., M. Shong, S. I. Taniguchi, K. Suzuki, C. Giuliani, G. Napolitano, J. Saito, M. Saji, B. Fiorentino, A. M. Reimold, D. S. Singer, and L. D. Kohn. 1998. Regulation of major histocompatibility class II gene expression in FRTL-5 thyrocytes: opposite effects of interferon and methimazole. Endocrinology 139:290-302.
30. Mozes, E., L. D. Kohn, F. Hakim, and D. S. Singer. 1993. Resistance of MHC class I-deficient mice to experimental systemic lupus erythematosus. Science 261:91-93.
31. Wenisch, C., D. Myskiw, A. Gessl, and W. Graninger. 1995. Circulating selecting, intercellular adhesion molecule-1, and vascular cell adhesion molecule-1 in hyperthyroidism. J Clin Endocrinol Metab 80:2122-2126.
32. Oren, R., Y. Maaravi, F. Karmeli, G. Kenet, L. Zeidel, A. Hubert, and R. Eliakim. 1997. Anti-thyroid drugs decrease mucosal damage in a rat model of experimental colitis. Aliment Pharmacol Ther 11:341-345.
33. Suzuki, K., A. Mori, K. J. Ishii, J. Saito, D. S. Singer, D. M. Klinman, P. R. Krause, and L. D. Kohn. 1999. Activation of target-tissue immune-recognition molecules by double-stranded polynucleotides. Proc Natl Acad Sci USA 96:2285-2290.
34. Gopalan, P. K., D. A. Jones, L. V. McIntire, and C. W. Smith. 1996. Cell adhesion under hydrodynamic flow conditions. In Current Protocols in Immunology. J. E. Coligan, A. M. Kruisbeek, D. H. Margulies, E. M. Shevach, and W. Strober, eds. J. Wiley, New York, p. 7.29.1-7.29.23.
35. Carlos, T., N. Kovach, B. Schwartz, M. Rosa, B. Newman, E. Wayner, C. Benjamin, L. Osborn, R. Lobb, and J. Harlan. 1991. Human monocytes bind to two cytokine-induced adhesive ligands on cultured human endothelial cells: endothelial-leukocyte adhesion molecule-1 and vascular cell adhesion molecule-1. Blood 77:2266-2271.
36. Zapolska-Downar, D., A. Zapolski-Downar, M. Markiewski, A. Ciechanowicz, M. Kaczmarczyk, and M. Naruszewicz. 2001. Selective inhibition by probucol of vascular cell adhesion molecule-1 (VCAM-1) expression in human vascular endothelial cells. Atherosclerosis 155:123-130.
37. Alon, R., H. Rossitier, X. Wang, T. A. Springer, and T. S. Kupper. 1994. Distinct cell surface ligands mediate T lymphocyte attachment and rolling on P- and E-selectin under physiological flow. J. Cell Biol. 127:1485-1495.
38. Alon, R., P. D. Kassner, M. W. Carr, E. B. Finger, M. E. Hemler, and T. A. Springer. 1995. The integrin VLA-4 supports tethering and rolling in flow on VCAM-1. J Cell Biol 128:1243-1253.
39. Ochi, H., J. Masuda, and M. A. Gimbrone. 2002. Hyperosmotic stimuli inhibit VCAM-1 expression in cultured endothelial cells via effects on interferon regulatory factor-1 expression and activity. Eur J Immunol 32:1821-1831.
40. Koo, S. W., K. A. Casper, K. B. Otto, A. K. Gira, and R. A. Swerlick. 2003. Iron chelators inhibit VCAM-1 expression in human dermal microvascular endothelial cells. J Invest Dermatol 120:871-879.
41. Ahmad, M., P. Theofanidis, and R. M. Medford. 1998. Role of activating protein-1 in the regulation of the vascular cell adhesion molecule-1 gene expression by tumor necrosis factor-alpha. J Biol Chem 273:4616-4621.
42. Umetani, M., C. Mataki, N. Minegishi, M. Yamamoto, T. Hamakubo, and T. Kodama. 2001. Function of GATA transcription factors in induction of endothelial vascular cell adhesion molecule-1 by tumor necrosis factor-alpha. Arterioscler Thromb Vasc Biol 21:917-922.

1. A method for inhibiting cell adhesion in a mammal which comprises administering to the mammal a pharmaceutical composition comprising a methimazole derivative or tautomeric cyclic thione or mixtures thereof in an amount effective for prevention, inhibition or suppression of cell adhesion.

2. A method for the treatment of diseases, disorders, conditions or symptoms mediated by cell adhesion in a mammal which comprises administering to the mammal a pharmaceutical composition comprising a methimazole derivative or tautomeric cyclic thione or mixtures thereof in an amount effective for prevention, inhibition or suppression of cell adhesion.

3. The method according to claim 1, wherein the disease or condition is one or more of acute lung injury, allergic rhinitis, Alzheimer's disease, arthritis, asthma, atherosclerosis, autoimmune glomerulonephritis, Behcet's disease, cancer, cerebral infarcts, chronic hepatitis, cirrhosis, cutaneous anaphylaxis reaction, cutaneous vasculitides, delayed type hypersensitivity reaction, diabetes, disseminated intravascular coagulation, eosinophilic granuloma of the lung, gastritis, giant cell arteritis, Graves' disease, haemorrhagic shock, hypertensive vascular disease, hypothyroidism, inflammatory bowel disease (Crohns' disease and ulcerative colitis), inflammatory dermatoses, intestinal infarcts, Kawasaki's disease (mucocutaneous lymph node syndrome), lymphoid interstitial pneumonia, malaria, meningitis, multiple sclerosis, myocardial infarcts, organ transplantation (host vs. graft and graft vs. host), polyarteritis nodosa, polymyositis/dermatomyositis, psoriasis, pulmonary infarcts, renal infarcts, reperfusion injury following ischemia, Rickettsial vasculitis, sarcoidosis, sepsis, sjogren's disease, stroke, systemic lupus erythematosus, thermal injury (burns), thromboangiitis obliterans (Buerger's disease), thrombosis, thyroiditis, tuberculosis, vasculitis, and Wegner's granulomatosis.

4. A method of preventing, inhibiting or suppressing cell adhesion in a mammal comprising the step of administering to the mammal a pharmaceutical composition comprising a methimazole derivative or tautomeric cyclic thione or mixtures thereof in an amount effective for prevention, inhibition or suppression of cell adhesion, wherein the cell adhesion is VCAM-1 mediated and a pharmaceutically acceptable carrier.

5. The method according to claim 4, wherein the cell adhesion is mediated by VCAM-1 and E-selectin.

6. The method according to claim 5, wherein the cell adhesion is IRF-1 dependent VCAM-1 mediated cell adhesion.

7. A method of preventing, inhibiting or suppressing cytokine-induced cell adhesion in a mammal comprising the step of administering to the mammal a pharmaceutical composition comprising a methimazole derivative or tautomeric cyclic thione or mixtures thereof in an amount effective for prevention, inhibition or suppression of cell adhesion, wherein the cell adhesion is VCAM-1 mediated and a pharmaceutically acceptable carrier.

8. The method according to claim 7, wherein the cell adhesion is mediated by VCAM-1 and E-selectin.

9. The method according to claim 8, wherein the cell adhesion is IRF-1 dependent VCAM-1 mediated cell adhesion.

10. The method according to claim 7, wherein the cytokine is TNF-alpha.

11. The method according to claims 1, 4 or 7, wherein the method is used for preventing, inhibiting or suppressing cell adhesion-associated inflammation.

12. The method according to claim 7 wherein the method is used for preventing, inhibiting or suppressing cytokine-induced cell adhesion-associated inflammation.

13. The method according to claims 1, 4 or 7, wherein the method is used for preventing, inhibiting or suppressing cell adhesion associated with immune or autoimmune responses.

14. The method according to claim 4, wherein the method is used to treat or prevent a disease selected from the group consisting of wherein the disease or condition is one or more of adult respiratory distress syndrome, AIDS, allergy conditions, arteriosclerosis, arthritis, asthma, atherosclerosis, cardiovascular diseases, detaching retina, harmful platelet aggregation, inflammation, inflammatory bowel diseases, multiple sclerosis, neoplastic diseases, ophthalmic inflammatory conditions, osteoarthritis, osteoporosis, psoriasis, regional enteritis, rejection after transplantation, reocclusion following thrombolysis, reperfusion injury, Sjogren's Syndrome, skin inflammatory diseases, systemic lupus erythematosus, thrombosis, Type I diabetes, thyroiditis and wounds.

15. The method according to claim 1, 4 or 7, wherein the pharmaceutical composition comprises a safe and effective amount of an active compound selected from: 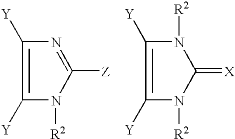
wherein Y is selected from the group consisting of H, C₁-C₄ alkyl, C₁-C₄ substituted alkyl, —NO₂, and the phenyl moiety: 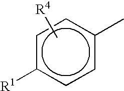
and wherein no more than one Y group in said active compound may be the phenyl moiety; R¹ is selected from the group consisting of H, —OH, C₁-C₄ alkyl, and C₁-C₄ substituted alkyl; R² is selected from the group consisting of H, C₁-C₄ alkyl and C₁-C₄ substituted alkyl; R³ is selected from the group consisting of H, C₁-C₄ alkyl, C₁-C₄ substituted alkyl, and —CH₂Ph; R⁴ is selected from the group consisting of H, C₁-C₄ alkyl, and C₁-C₄ substituted alkyl; X is selected from S and O; and Z is selected from —SR³, —OR³ and C₁-C₄ alkyl; and wherein at least two of the R² and R³ groups in said compound are C₁-C₄ alkyl when Y is not a phenyl moiety, and at least one Y is —NO₂ when Z is alkyl; and a pharmaceutically-acceptable carrier.

16. The method according to claim 15, wherein Z is selected from —SR³ and OR³.

17. The method according to claim 16, wherein Z is —SR³ and X is S.

18. The method according to claim 17, wherein Y is H.

19. The method according to claim 18, wherein R³ is C₁-C₄ alkyl.

20. The method according to claim 19, wherein R³ is methyl.

21. The method according to claim 20, wherein at least one of the R² groups is methyl.

22. The method according to claim 20, wherein both R² groups are methyl.

23. The method according to claim 15, wherein the active compound has the formula

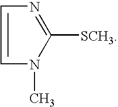

24. The method according to claim 15, wherein the active compound has the formula

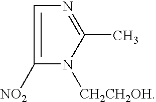

25. The method according to claim 15, wherein the active compound has the formula:

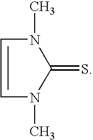

26. The method according to claim 15, wherein the active compound has the formula:

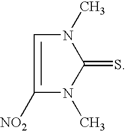

27. The method according to claim 15, wherein the active compound has the formula:

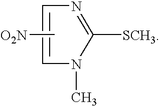

28. The method according to claim 17, wherein one of the Y groups is the phenyl moiety.

29. The method according to claim 26, wherein R¹ and R⁴ are H.

30. The method according to claim 27, wherein R³ is methyl and at least one of the R² groups is methyl.

31. The method according to claim 28, wherein R³ is H.

32. The method according to claim 29, wherein both R² groups are methyl.

33. The method according to claim 15, wherein the active compound is selected from the group consisting of:

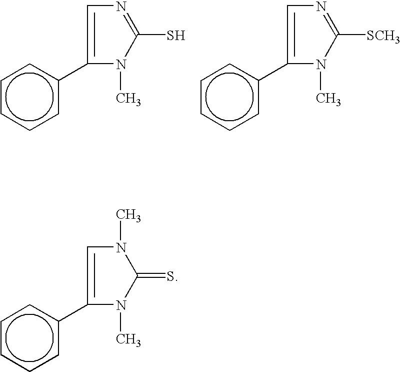

34. The method according to claim 15, wherein the pharmaceutical composition is in prodrug form.

35. The method according to claim 15, wherein the pharmaceutical composition comprises from about 0.01% to about 25% of the active compound and from about 75% to about 99.99% of the pharmaceutically-acceptable carrier.

36. The method according to claim 1, 4 or 7, wherein the pharmaceutical composition comprises a safe and effective amount of an active compound having the formula: 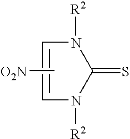
wherein R² is selected from the group consisting of H, C₁-C₄ alkyl and C₁-C₄ substituted alkyl.

37. The method according to claim 34, wherein the R² is selected from the group consisting of C₁-C₄ alkyl and C₁-C₄ substituted alkyl.

38. The method according to claim 35, wherein the R² is methyl.

39. The method according to claim 1, 4 or 7, wherein the pharmaceutical composition comprises a safe and effective amount of a compound selected from 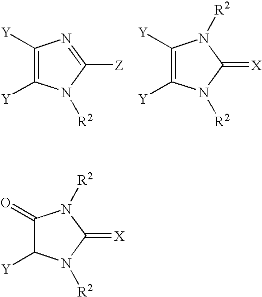
wherein Y is selected form the group consisting of H, C₁-C₄ alkyl, C₁-C₄ substituted alkyl, —NO₂, and the phenyl moiety 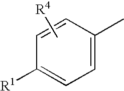
and wherein no more than one Y group is said active compound may be the phenyl moiety; R¹ is selected from the group consisting H, —OH, halogens, C₁-C₄ alkyl, C₁-C₄ substituted alkyl, C₁-C₄ ester and C₁-C₄ substituted ester; R² is selected from the group consisting of H, C₁-C₄ alkyl and C₁-C₄ substituted alkyl; R³ is selected from the group consisting of H, C₁-C₄ alkyl, C₁-C₄ substituted alkyl, and —CH₂Ph; R⁴ is selected from the group consisting of H, C₁-C₄ alkyl and C₁-C₄ substituted alkyl; X is selected from S and O; Z is selected from —SR³, —OR³, —S(O)R³, —SR³, and C₁-C₄ alkyl; and wherein at least two of the R² and R³ groups in said compound are C₁-C₄ alkyl when Y is not a phenyl moiety, and at least one Y is —NO₂, when Z is alkyl:
and a pharmaceutically-acceptable carrier.

40. The method according to claim 37, wherein the active compound is selected from the group consisting of 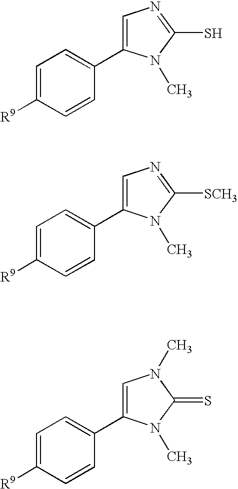
wherein R⁹ is selected from the group consisting of —OH, -M and —OOCCH₂M;
wherein M is selected from F, Cl, Br and I.

41. The method according to claim 38, wherein the active compound is selected from the group consisting of 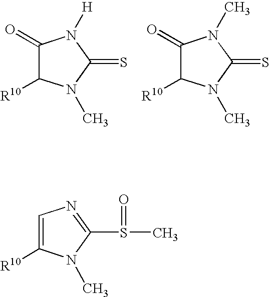
wherein R¹⁰ is selected from the group consisting of H, —NO₂, Ph, 4-HOPh and 4-MPh, wherein M is selected from F, Cl, Br and I.

42. A method of treating a condition involving vascular adhesion of leukocytes, comprising: (a) identifying a subject suspected of having a condition involving aberrant leukocyte adhesion to vascular endothelium; and (b) administering to the subject an amount of a pharmaceutical composition comprising a methimazole derivative or tautomeric cyclic thione or mixtures thereof sufficient to decrease cell surface expression of at least one of VCAM-I and E-selectin on endothelial cells, thereby reducing adhesion of leukocyte cells to vascular endothelium.

43. The method according to claim 40, wherein one or more additional active ingredients are combined with the composition of the present invention, either administered separately or in the same pharmaceutical composition, selected from the group comprising (a) VCAM-1 antagonists; (b) steroids; (c) immunosuppressants; (d) antihistamines; (e) non-steroidal anti-asthmatics; (f) non-steroidal antiinflammatory agents (NSAIDs); (g) cyclooxygenase-2 (COX-2) inhibitors; (h) inhibitors of phosphodiesterase type IV (PDE-IV); (i) antagonists of the chemokine receptors; (O) cholesterol lowering agents; (k) anti-diabetic agents; (1) preparations of interferon beta; (m) anticholinergic agents; and (n) antibiotics.

44. A method of screening for compounds capable of preventing, inhibiting or suppressing VCAM-1 mediated cell adhesion or preventing, inhibiting or suppressing cell adhesion-associated inflammation in a mammal, comprising:
(a) contacting the mammal with a test compound;
(b) measuring an effect of the test compound on leukocyte adhesion or migration or both; and
(c) determining whether the test compound is an inhibitor of the adhesion or migration activities or both of the leukocytes.

45. The method according to claim 42, wherein the measuring an effect of the test compound comprises one or more of measuring IRF-1 RNA expression levels; measuring IRF-1 protein expression levels; measuring IRF-1 dependent VCAM-1 promoter activation; measuring cytokine-increased IRF-1 RNA expression levels; measuring cytokine-increased IRF-1 protein expression levels; measuring cytokine-increased IRF-1 dependent VCAM-1 promoter activation; measuring TNF-alpha-increased IRF-1 RNA expression levels; measuring TNF-alpha-increased IRF-1 protein expression levels; and measuring TNF-alpha-increased IRF-1 dependent VCAM-1 promoter activation.

46. The method according to claim 42, wherein the measuring an effect of the test compound comprises one or more of measuring VCAM-1 RNA expression levels; measuring cytokine-increased VCAM-1 protein expression levels; measuring cytokine-increased VCAM-1 promoter activation measuring TNF-alpha-increased VCAM-1 RNA expression levels; measuring TNF-alpha increased VCAM-1 protein expression levels; and measuring TNF-alpha increased VCAM-1 promoter activation.

47. A method of screening for compounds capable of cell adhesion inhibitory activity in VCAM-1 expressing cells, comprising:
(a) contacting VCAM-1-expressing cells with a test compound;
(b) contacting the VCAM-1-expressing cells to VCAM-1 ligand expressing cells;
(c) measuring an effect of the test compound on binding of the VCAM-1-expressing cells to VCAM-1 ligand expressing cells; and
(d) determining whether the test compound is an inhibitor of the binding activities of the VCAM-1-expressing cells to VCAM-1 ligand expressing cells.

48. The method according to claim 45, comprising the further step prior to, concurrently with or subsequently to addition of the test compound, of contacting VCAM-1-expressing cells with a cytokine capable of inducing expression of VCAM-1.

49. The method according to claim 46, wherein the cytokine is TNF-alpha.

50. The method according to claim 47, wherein the VCAM-1-expressing cells are selected from the group comprising nonimmune target tissue cells, endothelial cells, and epithelial cells.

51. The method according to claim 47, wherein the VCAM-1-expressing cells are human aorta endothelial cells (HAEC).

52. The method according to claim 47, wherein the VCAM-1 ligand expressing cells are leukocytes.

53. The method according to claim 47, wherein the cells are allowed to remain in contact for at least 30 minutes.


SEQUENCE LISTING (7 sequences)

SEQ ID NO: 1 - DNA, 31 bp, Artificial Sequence
  Primer for VCAM-1
gactccgtctcattgacttgcagcaccacag

SEQ ID NO: 2 - DNA, 31 bp, Artificial Sequence
  Primer for VCAM-1
atactcccgcatccttcaactgggcctttcg

SEQ ID NO: 3 - DNA, 28 bp, Artificial Sequence
  Primer for E-Selectin
gtgcagccattcccctgctggagagttc

SEQ ID NO: 4 - DNA, 28 bp, Artificial Sequence
  Primer for E-Selectin
gggccagagacccgaggagagttatctg

SEQ ID NO: 5 - DNA, 33 bp, Artificial Sequence
  Primer for ICAM-1
ctcaggtatccatccatcccagagaagccttcc

SEQ ID NO: 6 - DNA, 33 bp, Artificial Sequence
  Primer for ICAM-1
cccttgagttttatggcctcctcctgagccttc

SEQ ID NO: 7 - DNA, 22 bp, Artificial Sequence
  NF- kappaB probe
agttgaggggactttcccaggc